(19) United States
(12) Patent Application Publication
(10) Pub. No.: US 2011/0165143 A1
(43) Pub. Date: Jul. 7, 2011

(54) MODULATION OF CASPASES AND THERAPEUTICAL APPLICATIONS

(75) Inventors: Chuan-Yuan Li, Greenwood Village, CO (US); Fang Li, Englewood, CO (US)

(73) Assignee: The Regents of the University of Colorado, a body corporate, Denver, CO (US)

(21) Appl. No.: 12/985,324

(22) Filed: Jan. 5, 2011

Related U.S. Application Data
(60) Provisional application No. 61/292,821, filed on Jan. 6, 2010.
(60) Provisional application No. 61/330,008, filed on Apr. 30, 2010.

(51) Int. Cl. A61K 38/46; A61K 38/06; A61P 35/00; A61P 17/02; C12N 5/071

(52) U.S. Cl. 424/94.65; 514/19.3; 435/377

(57) ABSTRACT

The present invention provides caspase modulators in a wide variety of therapeutic applications. Some aspects of the present invention provide methods for using caspase modulators in treating tumor, promoting wound healing, and producing induced pluripotent stem cells.

### CROSS-REFERENCE TO RELATED APPLICATIONS

This application claims the priority benefit of U.S. Provisional Application Nos. 61/292,821, filed Jan. 6, 2010, and 61/330,008, filed Apr. 30, 2010, which are incorporated herein by reference in their entirety.

### STATEMENT REGARDING FEDERALLY FUNDED RESEARCH

This invention was made with government support under grant numbers CA131408 and CA136748 awarded by the National Institutes of Health. The government has certain rights in the invention.

### FIELD OF THE INVENTION

The present invention relates to using caspase modulators in a wide variety of therapeutic applications. In particular, the present invention relates to using caspase modulators in treating tumor, promoting wound healing, and producing induced pluripotent stem cells.

### BACKGROUND OF THE INVENTION

Caspases are a family of cysteine proteases that play essential roles in various cell functions. Some of the most important roles of caspases are believed to be their role in apoptosis, necrosis, and inflammation. While some of the uses of caspase modulators have been identified, there are still a wide variety of unknown applications for caspase modulators.

Since the role of caspases in cell functions are not fully understood, there is a continuing need to identify other roles of caspases in cellular functions in order to identify and use caspase modulators in heretofore unknown therapeutic uses.

### SUMMARY OF THE INVENTION

Apoptosis is generally recognized to be a process for multi-cellular organisms to remove unwanted or damaged cells. In cancer treatment, apoptosis is believed to be a key mechanism through which cytotoxic agents, including radiation and anti-cancer drugs, kill tumor cells. It has been shown that while radiation treatment and anti-cancer drug therapies kill tumor cells, these treatments also appear to initiate a process called “accelerated repopulation.” In this process, surviving tumor cells rapidly proliferate, repopulating at a significantly accelerated pace. Further confounding cancer treatment is that radiation and chemotherapy regimens incorporate necessary intervals in treatment, to allow for the recovery, survival, and repopulation of normal cellular tissue. It is believed that these breaks in treatment, paired with “accelerated repopulation,” allow for rapid growth of surviving tumor cells. In fact, this repopulation of surviving tumor cells is believed to be a common cause of cancer treatment failure.

Thus, some aspects of the invention provide methods for treating tumor in a subject by administering a growth stimulating signal inhibitor. In some embodiments, such methods include:
administering a therapeutically effective amount of a cytotoxic agent to the subject having a tumor to cause apoptosis of the tumor cells; and
administering a growth stimulating signal inhibitor to the subject to inhibit the growth stimulating signal released by the apoptotic tumor cells to reduce the amount of new tumor cell generation in the subject.

In some instances, the cytotoxic agent comprises a chemotherapeutic agent. While in other instances, the cytotoxic agent comprises electromagnetic radiation. Yet still in other instances, the cytotoxic agent can comprise both a chemotherapeutic agent and an electromagnetic radiation.

In some embodiments, the growth stimulating signal inhibitor is administered about 144 hours (i.e., 6 days) or less, typically about 5 days or less, often about 4 days or less, more often about 3 days or less, and most often about 2 days or less after the administration of the cytotoxic agent.

Alternatively, the growth stimulating signal inhibitor is administered from about 1 hour to about 144 hours, typically from about 1 hour to about 120 hours, often from about 1 hour to 96 hours, more often from about 1 hour to 72 hours, and most often from about 1 hour to about 48 hours after the administration of the cytotoxic agent.

Yet in other embodiments, the growth stimulating signal inhibitor comprises a caspase inhibitor. Within these embodiments, in some instances the growth stimulating signal inhibitor inhibits caspase-2, caspase-3, caspase-6, caspase-7, caspase-8, caspase-9, or a combination thereof. Exemplary caspase inhibitors that are useful in methods of the invention include, but are not limited to, zVAD-fmk, z-DEVD-fmk, ac-YVAD-cmk DEVD-cho, and other known competitive or non-competitive caspase inhibitors. It should be appreciated that the growth stimulating signal inhibitor can comprise a mixture of two or more different caspase inhibitors.

Still in other embodiments, the tumor is breast cancer, prostate cancer, colon cancer, melanoma, liver cancer, leukemia, lymphoma, and other solid tumors and blood-borne malignancies, or a combination thereof.

Preferably the growth stimulating signal inhibitor is non-cytotoxic.

The growth stimulating signal inhibitor can be administered parenterally, orally, nasally, buccally, intravenously, intramuscularly, subcutaneously, intrathecally, transdermally, by osmotic pump, by inhalation, or a combination thereof.

In some instances, the growth stimulating signal inhibitor is administered at or near the tumor site.

While it may appear that apoptosis is unrelated to wound healing, the present inventors have discovered that a similar mechanism of repopulating cells is responsible for damaged tissue repair. It is well known that some lower organisms (e.g., salamanders) possess the remarkable ability to completely regenerate whole amputated limbs. In contrast, more advanced organisms such as humans can only partially replace damaged organs (e.g., liver regeneration). Studies have shown that tissue regeneration is a complicated process involving many different cell types. It is generally believed that stem/progenitor cells in and around damaged tissues play critical roles in the regenerative process. These stem/progenitor cells are thought to be mobilized by secreted factors from the damaged tissues, presumably released during the inflammatory process ensuing initial tissue damage. The mobilized stem/progenitor cells are believed to migrate to the damaged site, proliferate, differentiate and eventually replace the damaged tissues.

One of the puzzles in tissue regeneration/wound healing is how the stem/progenitor cells are stimulated/mobilized by damaged tissues. Previous studies have focused on the roles of immunoeffector cells (e.g., macrophages) that are activated and secrete cytokines/growth-factors that promote wound healing/tissue regeneration.

Surprisingly and unexpectedly, the present inventors have discovered that dying cells in the wounded tissues can send signals directly to stimulate the proliferation of stem/progenitor cells.

Accordingly other aspects of the invention provides methods for treating a wound in a subject. Such methods typically comprise administering a therapeutically effective amount of a wound healing composition to the subject to treat the wound. The wound healing composition comprises:
(i) an exogenous caspase or a moiety having a similar protease activity;
(ii) a caspase activity promoter, wherein the caspase activity promoter increases the production or activity of one or more caspases; or
(iii) a combination of (i) and (ii)
   wherein said method increases the rate of wound healing relative to the wound that is not treated with the wound healing composition.

In some embodiments, the caspase activity promoter increases the production or activity of caspase-3, caspase-7, caspase-8, or a combination thereof.

Yet another aspect of the invention provides methods for increasing the rate of wound healing in a subject. Such methods comprise increasing the production or activity of one or more caspases at or near the wound site by administering a wound healing composition described herein. The caspase activity promoter often increases the production or activity of caspase-3, caspase-7, caspase-8, or a combination thereof.

Surprisingly and unexpectedly, the present inventors have also found that caspases also play a role in producing induced pluripotent stem cells (iPSCs) from differentiated cells. In particular, the present inventors have discovered that caspases 3 and 8, two proteases associated with apoptotic cell death, are involved in induction of iPSCs from differentiated cells such as human fibroblast cells. It was discovered that activation of caspases 3 and 8 occurs soon after transduction of iPS-inducing transcription factors. Without being bound by any theory, it is believed that Oct-4, a key iPSC transcription factor, is involved in the activation. Moreover, it is believed that caspases play a role in reducing or overcoming epigenetic barriers to facilitate nuclear reprogramming in the iPSC derivation process.

Thus, other aspects of the invention provide methods for producing induced pluripotent stem cells (iPSC) from differentiated cells. Such methods comprise subjecting differentiated cells to conditions sufficient to produce iPSC in the presence of:
(i) exogenous caspase-3, caspase-8, or a combination thereof;
(ii) a caspase activity promoter, wherein the caspase activity promoter increases the production or activity of caspase-3, caspase-8, or a combination thereof; or
(iii) a combination of (i) and (ii).

In some embodiments, differentiated cells are human fibroblast cells, keratinocytes, or a combination thereof.

### BRIEF DESCRIPTION OF THE DRAWINGS

FIG. 1 is a graph showing growth of various Fluc-labeled human tumor cells (about 1000 each) plated onto human IMR-90 fibroblast cells (about 2×10⁵) lethally treated with x-rays (10 Gy) (top panel) or heated (43° C. for 30 minutes) (lower panel).

FIG. 2a is a graph showing the effect of caspase 3 deficiency in vitro. After lethal irradiation (10 Gy), wild type and casp3−/− MEF were plated into 12-well plates at a density of 2×10⁵ cells/well. Twenty-four hours later, about 1000 4T1Fluc cells were plated into each well and the growth of these cells were followed by bioluminescence imaging. The data plotted were those from day 10 after plating. In all three groups, the difference between wild type and casp3−/− MEF cells are statistically significant (P<0.01, t-test). Error bars represent standard error of the mean (SEM, n=3).

FIG. 2b is a graph and electrophoresis plate showing the effect of caspase 3 knockdown in vitro. 4T1 cells stably transduced with a shRNA minigene targeted to murine caspase 3 were evaluated for their role to support a small number of 4T1Fluc tumor cell growth. About 1000 4T1Luc cells were mixed with 2×10⁵ 4T1 or 4T1 cells with caspase 3 knockdown (4T1-casp3kn) and then monitored for their growth in vitro. Data from 4 independent anti-caspase 3 shRNA (casp3kn)-transduced 4T1 clones as well as a control line transduced with a scrambled shRNA minigene ar shown. The difference between the control groups and the casp3kn groups are statistically significant (P<0.01 between each of the control and each of the caspase 3 knock down clones, n=3, t-test). The pseudo-colored bioluminescent images were taken at day 7.

FIG. 3 is a graph showing growth of Fluc-labeled MCF-7Fluc cells (about 1000) when plated either alone, with lethally irradiated parental MCF-7 cells, which does not express caspase 3, or with MCF7-C3, which express caspase 3 (C3 indicates exogenous caspase 3 gene). Lethally irradiated cells numbered around 2×10⁵.

FIG. 4 is a graph showing the effect of a caspase inhibitor z-VAD-fmk on 4T1Fluc tumor cell growth when plated with lethally irradiated NIH3T3 cells. About 1000 4T1Fluc cells were plated either alone, with non-irradiated NIH3T3 cells, or with lethally irradiated NIH3T3 cells plus a pan-caspase inhibitor z-VAD-fmk.

FIG. 5 shows the result of Western blot analyses of key proteins in the apoptotic pathway. 4T1 cells exposed to various radiation doses were lysed 24 hrs after exposure. The lysates were then blocked for various protein expression. Cytosolic cytochrome c was analyzed through the use of a digitonin protocol.

### DETAILED DESCRIPTION OF THE INVENTION

### Inhibition of Growth Stimulation Signal

How tumors respond to cytotoxic agents is affected by many factors. “Cytotoxic agent” refers to any extracellular agent that causes cell death. Exemplary cytotoxic agents include radiation (e.g., x-ray), heat, and chemicals (e.g., drugs that are used to treat cancer). It has been shown that cancer cells respond to cytotoxic agents by activating a variety of molecular signaling pathways such as DNA repair, cell cycle arrest, free radical scavenging, and kinase signaling cascades such as Ras/RAF and PKC, etc. Most of these pathways were activated to defend against or repair damage to various vital cellular targets. It is generally believed that cancer cells that successfully protected their critical components will survive and repopulate the tumor. In contrast, cells with overwhelmed repair system will suffer excessive damage and die. Although it is obvious that overall response of a tumor to cytotoxic therapy will be determined by the interplay between cellular life and death within the tumor, little is currently understood about the details of the interplay.

Cytotoxic therapy-induced cancer cell death can occur in a number of different ways. These include necrosis, autophagy, senescence, and apoptosis. Among these, apoptosis, or programmed cell death, is currently the most extensively studied and well characterized at both molecular and cellular levels. In adult mammals, the purpose of apoptosis is believed to be a mechanism through which unwanted or damaged cells are removed rapidly and efficiently.

Cytotoxic therapy, such as radiation therapy and/or chemotherapy, results in a massive amount of cell death, typically in the order of three to ten logs of cell killing. It is generally assumed that most dying cells are absorbed by “scavenger” cells like macrophages or other surviving cells in the vicinity. The small number of surviving tumor cells, if any, is believed to be then gradually and slowly proliferate and re-establish the tumor. However, studies that originated more than 40 years ago have indicated that tumors respond to radiotherapy by initiating a process called “accelerated repopulation”. In this process, the few surviving tumor cells that survived radiotherapy or chemotherapy can rapidly repopulate the tumor by proliferating at a significantly accelerated pace. This phenomenon, for which little is understood at the molecular level, has played an important role in modern radiotherapy and chemotherapy. The main legacy of this discovery is that radiation therapy is now administered at a schedule that is as intensive as patients can tolerate to avoid the rapid repopulation that may occur when the time interval between two treatments are too long. This treatment principle has become a basic tenet in conventional clinical cancer treatment.

Numerous efforts have been attempted to understand the molecular mechanism of tumor response, including tumor repopulation, after cytotoxic therapy. For example, it has been discovered that cytotoxic treatment can induce the activation of EGFR signaling in tumor cells. It has also been reported that angiogenesis-promoting factors such as VEGF, IGFR-I, HGF/c-Met, etc, are induced by radiation therapy. Radiation-induced up-regulation of angiogenic activities in tumors has also been linked with activation of upstream transcriptional factors such as HIF-1. Recent studies have also indicated the importance of macrophages in facilitating tumor recovery after radiation. Furthermore, the integrity of endothelial cells has been implicated in tumor response to radiotherapy. These studies, while providing how radiation-induced signaling events can facilitate the re-growth of tumors, still fall short in presenting a clear mechanistic explanation for the phenomenon of accelerated repopulation in tumors undergoing cytotoxic therapy.

Some aspects of the invention are based on the discovery by the present inventors of a molecular signaling pathway integral in the process of “accelerated repopulation.” With this discovery, the present inventors have developed an approach to significantly reduce or prevent tumor repopulation and metastasis, thereby enhancing the effectiveness of cancer treatment. Such a discovery can also be used in other applications including tissue damage repair. This discovery by the present inventors differs significantly from the widely held view that cell death is mostly a passive event. Surprisingly and unexpectedly, it was discovered by the present inventors that intratumoral dying cells play a significant role in promoting the rapid repopulation of tumors from a small number of live tumor cells. In addition, it was discovered that in some instances caspase-3, a cysteine protease involved in the “execution” phase of cellular apoptosis, is one of the key regulators of growth-promoting signals generated from the dying cells. This caspase-mediated, cell-death stimulated tumor repopulation mechanism discovered by the present inventors has profound implications that go far beyond cancer biology and cancer therapy.

As disclosed herein, the present inventors have discovered that apoptotic cells release growth stimulating signal(s) that stimulate the proliferation of progenitor cells. In some instances, caspases, such as caspase-3, was found to play a role in this signaling event. It was discovered that in some cases activated Caspase-3 results in a rapid proliferation of surviving cancerous tumor cells. In particular, it was discovered that administering a cytotoxic agent (e.g., radiation or a chemical cytotoxic agent) to tumor cells activates the growth stimulating signal. Thus, by decreasing the growth stimulating signal expression (e.g., using shRNA) or by inhibiting the activity of the growth stimulating signal, cellular growth of tumor cells or repopulation of tumor cells treated with a cytotoxic agent can be significantly reduced or prevented. Exemplary growth stimulating signals include, but are not limited to, caspases (e.g., caspase-3, caspase-7, caspase-2, caspase-8, caspase-6, and caspase-9), and calcium-independent phospholipase A2, protein kinase delta, both of which could be activated by caspase mediated cleavage. While methods of the invention can include inhibiting the activity of or reducing the expression of any growth stimulating signal(s) disclosed herein, for the sake of clarity and brevity, the present invention will now be disclosed in reference to inhibiting the activity of or reducing the expression of caspase-3.

In fact, in some particular embodiments, methods of the invention include using a caspase-3 inhibitor or a caspase-3 expression inhibitor (e.g., shRNA) to significantly reduce or prevent tumor repopulation and metastasis following radiotherapy and/or chemotherapy. Such methods significantly improve the prognosis of cancer patients.

Some particular aspects of the invention provide methods for treating tumor in a subject. Such methods typically comprise:
administering a therapeutically effective amount of a cytotoxic agent to the subject in need of such a treatment; and
administering a therapeutically effective amount of a growth stimulating signal inhibitor to the subject to inhibit the growth stimulating signal released by the apoptotic tumor cells or to inhibit the expression of the growth stimulating signal to reduce the amount of new tumor cell generation in the subject.
   The effectiveness of the treatment can be readily determined by one skilled in the art, for example, by in vitro experiment comparing the effects of tumor cells treated with a cytotoxic agent followed by administration of a growth stimulating signal inhibitor relative to those not treated with the same growth stimulating signal inhibitor. In vivo experiments involving patients who are given a growth stimulating signal inhibitor after treatment with a cytotoxic agent and those receiving placebo after treatment with a cytotoxic agent can also be used to determine whether a particular growth stimulating signal inhibitor is effective in reducing or preventing the growth of new tumor cells.

As used herein, administering “a therapeutically effective amount” of a cytotoxic agent means the amount of cytotoxic agent that, when administered to the subject, is sufficient to cause apoptosis of at least a portion of the tumor cells. As used herein administering “a therapeutically effective amount” of a growth signal inhibitor means the amount of a compound that, when administered to the subject, is sufficient to reduce or prevent repopulation of the tumor cells, i.e., the number of new tumor cells. Typically, the “therapeutically effective amount” of the growth signal inhibitor will vary depending on the compound, and the age, weight, etc., of the subject to be treated. As used herein, the term “reduce the amount of new tumor cell generation” means decreasing the number of new tumor cells generated compared to untreated apoptotic tumor cells or preventing new tumor cell generation. It should be appreciated that the “growth stimulating signal inhibitor” can inhibit the activity of the growth stimulating signal or can inhibit expression (e.g., by inhibiting translation or transcription) of the growth stimulating signal.

In some embodiments, the cytotoxic agent comprises a chemotherapeutic agent. Yet in other embodiments, the cytotoxic agent comprises electromagnetic radiation, such as x-ray radiation.

In some instances, the growth stimulating signal inhibitor is administered about 144 hours (6 days) or less, often 4 days or less, more often 2 days or less, and still more often 24 hours or less, after the administration of the cytotoxic agent. Alternatively, the growth stimulating signal inhibitor is administered from about 1 hour to about 144 hours, typically from 1 hour to about 96 hours, often 1 hour to 48 hours, and more often 1 hour to 24 hours, after the administration of the cytotoxic agent.

Still in other embodiments, the growth stimulating signal inhibitor comprises a caspase inhibitor. Within these embodiments, in some instances the caspase inhibitor inhibits caspase-3, caspase-7, caspase 2, caspase 8, caspase 6, caspase 9, or a combination thereof. Specific non-limiting examples of useful caspase inhibitors of the invention include zVAD-fmk, z-DEVD-fmk, ac-YVAD-cmk DEVD-cho, and other known competitive and non-competitive caspase inhibitors. Methods of the invention can also include administering a mixture of such growth stimulating signal inhibitors.

Methods of the invention can be used to treat a wide variety of tumors including solid tumors and blood-borne malignancies. Exemplary non-limiting tumors that can be treated by methods of the invention include breast cancer, prostate cancer, colon cancer, melanoma, liver cancer, leukemia, lymphoma, and other solid tumors and blood-borne malignancies. Methods of the invention can also be used to treat multiple cancers, for example, in subjects that have more than one type of tumors (e.g., after cancer has spread to other organs).

It should be appreciated that the growth stimulating signal inhibitor is typically non-cytotoxic. Thus, the growth stimulating signal inhibitor does not directly cause any significant apoptosis of tumor cells.

The growth stimulating signal inhibitor can be administered by any method of administration known to one skilled in the art including, but not limited to, parenterally, orally, nasally, buccally, intravenously, intramuscularly, subcutaneously, intrathecally, transdermally, by osmotic pump, by inhalation, or a combination thereof.

The growth stimulating signal inhibitor can be administered at or near the tumor site, for example, by injection. Or it can be administered systemically. However, it should be appreciated that administration at or near the tumor site can often result in a significantly reduced side-effect and/or increased efficacy.

Tumors can be benign or malignant. Malignant tumors are called cancer. Accordingly, some aspects of the invention provide methods for treating cancer in a subject. Such methods comprise:
administering a therapeutically effective amount of a cytotoxic agent to the subject in need of such a treatment; and
administering a therapeutically effective amount of a growth stimulating signal inhibitor to the subject.

Yet other aspects of the invention include methods for reducing the number of tumor cell regeneration in a subject after a radiotherapy or a chemotherapy. Such methods include administering a caspase-3 inhibitor to the subject about 144 hours or less after the radiotherapy or the chemotherapy.

Still other aspects of the invention include methods for increasing the effectiveness of a radiotherapy or a chemotherapy in a subject having a tumor comprising reducing the number of tumor cell regeneration after a radiotherapy or a chemotherapy by administering a growth signal inhibitor to the subject about 144 hours or less after the radiotherapy or the chemotherapy. As used herein, the term “reducing the number of tumor cell regeneration” includes reducing the number of or completely preventing regeneration of tumor cells. Without being bound by any theory, it is believed that such methods typically inhibit the growth stimulation signal released by apoptotic tumor cells such that the number of tumor cell regeneration is substantially reduced compared to number of tumor cell regenerated without administration of the growth signal inhibitor. As used herein, the term “inhibiting the growth stimulation signal” includes preventing or reducing the activity or the expression of the growth stimulation signal. In some embodiments, the number of tumor cell regenerated is about 80% or less, typically about 50% or less, often about 30% or less, and more often about 10% or less relative or compare to the number of tumor cells regenerated without administration of the growth signal inhibitor.

### Wound Healing

The ability to regenerate damaged tissues is a common characteristic of multi-cellular organisms. The present inventors have discovered that surprisingly and unexpectedly apoptotic cell death plays a significant role in promoting tissue regeneration. As discussed herein, apoptotic cells release potent growth signals to stimulate the proliferation of progenitor/stem cells. One of the key players in this process is caspase 3, a protease activated in the execution phase of apoptosis. Mice with genetic knockout of caspase 3 are deficient in skin wound healing and in liver regeneration during partial liver hepatectomy. Prostaglandin E₂, a known promoter of stem/progenitor cell proliferation and tissue regeneration, acts downstream of caspase 3. The present inventors have discovered that caspases also promote tissue regeneration in mammalian organisms.

The ability to repair damaged tissues is essential for metazoan organisms. Lower organisms (e.g., salamanders) possess the remarkable ability to completely regenerate whole amputated limbs. In contrast, more advanced organisms such as humans can only partially replace damaged organs (e.g., liver regeneration). Studies have shown that tissue regeneration is a complicated process involving the coordinated efforts by many different cell types. It is believed that stem/progenitor cells in and around damaged tissues play critical roles in the regenerative process. These stem/progenitor cells are believed to be mobilized by signaling factors that are secreted from the damaged tissues. Without being bound by any theory, these signaling factors are believed to be released during the inflammatory process ensuing initial tissue damage. Subsequently stem/progenitor cells are believed to migrate to the damaged site, proliferate, differentiate and eventually replace the damaged tissues.

The present inventors have discovered that dying cells in the wounded tissues send signals to stimulate the proliferation of stem/progenitor cells. Experiments using irradiated mouse embryonic fibroblasts to simulate dying cells in wounded tissues demonstrated that in co-culture systems, dying mouse embryonic fibroblast cells significantly stimulated the proliferation of murine epidermal keratinocyte progenitor cells (EKP cells), neural stem cells (NSC), and mesenchymal stem cells (MSC). A similar growth-promoting property was observed from a variety of lethally irradiated human and mouse cells indicating that growth promoting ability towards stem/progenitor cells is a general property of dying mammalian cells.

The ability of dying cells to support the proliferation of stem/progenitor cells was also confirmed in vivo. When lethally irradiated mouse embryonic fibroblast (MEF) cells were co-injected subcutaneously with firefly luciferase (Fluc)-labeled mouse epidermal keratinocyte stem/progenitor cells (EKP-Fluc) into nude mice, the growth of these cells was significantly enhanced when compared with that of the EKP-Fluc cells injected alone. In fact, cellular growth was only observed for EKP-Fluc cells that were co-injected with irradiated MEF cells. For EKP-Fluc cells that were injected alone, no growth was observed.

Additional in vivo evidence of dying cells promoting or stimulating stem/progenitor cell proliferation came from experiments where EKP-Fluc cells were injected subcutaneously into lethally irradiated (18 Gy) and non-irradiated hind legs of nude mice. Experiments by the present inventors showed a clear growth-promoting effect of wounded tissues. EKP-Fluc cells injected into irradiated hind legs showed significant initial growth in the first week. Although cellular signals subsequently weakened, they persisted beyond day 103 after injection indicating long-term engraftment. In comparison, EKP-Fluc cells injected into non-irradiated left hind legs showed minimal proliferation and rapid signal attenuation afterwards. The irradiated right hind legs consistently demonstrated over 10 fold higher signal strength (beyond day 12) than the non-irradiated left hind legs. These results show that damaged and dying tissues stimulate proliferation of stem/progenitor cells.

Because of the regenerative capability among different tissues, the present inventors theorized that the factor(s) regulating cell death-stimulated stem/progenitor cell proliferation is derived from a common process operating in dying cells. In particular, the present inventors theorized that apoptosis, one of the major mechanisms of cell death in many different types of mammalian cells, is involved in stimulating stem/progenitor cell proliferation. Without being bound by any theory, it is believed that the molecular machinery coordinating the apoptotic process play a key role in initiating the paracrine cascades that leads to the stimulation of stem/progenitor cell growth.

Since caspases are proteases that function as the nexus of the apoptotic process, it is believed that these enzymes are involved in cell death-stimulated progenitor/stem cell proliferation. This was confirmed by experiments performed by the present inventors. In one particular experiment, the ability of lethally irradiated caspase 3-deficient (casp3−/−) and caspase 7-deficient (casp7−/−) mouse embryonic fibroblasts (MEF) were evaluated. Results indicated that compared with wild-type fibroblasts, both lethally irradiated casp3−/− and casp7−/− fibroblasts showed deficiencies in stimulating stem/progenitor cell growth. In addition, double deficiencies in caspase 3 and caspase 7 caused further attenuation in the ability of irradiated MEF cells to promote stem/progenitor cell growth, consistent with the overlapping functions of caspases 3 and 7.

The importance of caspase 3 was also examined in vivo. When lethally irradiated wild type and casp3−/− MEF were co-injected with EKP-Fluc progenitor cells, wild type MEF showed significantly higher efficiency in supporting EKP cellular growth than casp3−/− MEF cells further confirming the role of caspase 3 in mediating the paracrine growth stimulating properties of dying cells. Similar results were also obtained when lethally irradiated wild type and casp7−/− MEF cells were co-injected with EKP-Fluc cells, albeit the difference was smaller than those observed between casp3−/− and wild type MEF cells.

The effect of caspase 3 was also examined in a radiation-induced wound healing model by use of transgenic knockout mice. EKP-Fluc cells were injected subcutaneously into irradiated and non-irradiated hind legs of mice. Data indicated that irradiated tissues in wild type mice (in C57BL/6 background) showed significantly more capacity to promote EKP-Fluc cellular growth compared to irradiated tissues in casp3−/− mice (also in C57BL/6 background) towards the injected EKP-Luc cells during the observation period. These data again confirmed the role of caspase 3 in cell death-induced stimulation of stem/progenitor cell growth. Similar results were also obtained for casp7−/− mice indicating that caspase 7 also participates in the same process.

The role of caspase 3 was further demonstrated from an experimental tissue regeneration model. In this experiment, lethally irradiated wild type or casp3−/− mouse embryonic fibroblasts (mixed with basement membrane extract) were placed into silicone cylinders. The resulting cylinders were subcutaneously implanted into nude mice. After two weeks, the mice were sacrificed and the implanted cylinders were removed and host cellular growth was quantified by measuring the amount of host vascular growth into the cylinders. Data from this experiment showed that while irradiated wild type MEF cells induced significant vascular growth, irradiated casp3−/− MEF cells induced minimal vascular growth. In fact, the level of vascular growth in cylinders containing irradiated casp3−/− MEF cells was close to that of the negative PBS (phosphate-buffered saline) control indicating a significant role for caspase 3 in mediating cell death induced tissue regeneration. Similar results were obtained in a modified assay where experiments were done in transgenic GFP (green fluorescence protein)-expressing mouse. Here again, measuring gross host tissue growth into the silicone cylinders demonstrated a significant role of caspase 3 in mediating cell death induced tissue regeneration.

Two additional experiments were conducted in vivo. In the first experiment, the effects of caspase 3 on the ability of mice to heal excision wounds in the skin was examined. Excision wounds were created in wild type and casp3−/− mice in the C57BL/6 background through punch biopsy. The rates of wound healing in these mice were then quantified by measuring wound diameters. Results of the experiment showed that in caspase 3-deficient mice, the capacity to repair dorsal skin wounds were significantly compromised when compared with wild type mice. In fact, all the wounds in wild type mice were healed by day 9. In contrast, the time for complete wound closure in casp3−/− mice was 14 days. Immunofluorescence analyses were conducted to visualize the rate of wound closure through staining of the skin wound tissues at different time points using an antibody against cytokeratin 14, which is a marker for skin epithelial cells, a key cell type for basal layer formation during epidermal closure. The immunofluorescence staining unequivocally showed that skin epithelium recovery around the wound was significantly attenuated in caspase 3-deficient mice. By day 6, when there was already complete epidermal closure in wild type mice as demonstrated by continuous cytokeratin 14 staining across the wound gap, there was little signs of closure in the wound gap of caspase 3-deficient mice. In addition to cytokeratin 14 staining data, immunofluorescence data from cytokeratin 6 staining, which is specific for keratinocytes (and usually used to evaluate epidermal cellular migration), showed a similar pattern of staining.

In addition to defects in skin epithelial cell proliferation and migration, staining of BrdU, which was administered to mice shortly before their sacrifice, was also significantly attenuated in casp3−/− mice. Because BrdU incorporation is a direct indicator of DNA synthesis during cellular proliferation, these data are consistent with an overall defect in cellular proliferation in casp3−/− mice that should occur during normal skin wound healing. Quantification of representative fields indicated that there is at least a 10-fold reduction in BrdU incorporation in casp3−/− mice on the first day after skin biopsy. Experiments carried out in casp7−/− mice showed a similar defect in skin wound healing and BrdU incorporation.

In another experiment, the effects of caspase 3 on liver regeneration in a well-established partial hepatectomy model in mice was examined. In this model, two of the four lobes of liver (i.e., median and left lateral lobes, which account for about ⅔ of total liver weight) were surgically removed from wild type and casp3−/− mice, and the mice were allowed to recover. When measured in terms of liver weight, wild type mice showed a remarkable ability to regenerate lost liver tissue as expected. By day 10, the liver weight reached about 88% of pre-surgery weight in wild type mice. In contrast, liver weight in caspase 3−/− mice remained at only 60% of pre-surgery level indicating a significant defect in liver regeneration. Quantification of BrdU staining indicated that there was at least 50% reduction in BrdU staining in casp3−/− mice compared to wild type mice further confirming a defect in cellular proliferation in regenerating livers in caspase 3−/− mice. Similar defects in liver regeneration were also observed in casp7−/− mice. These results clearly indicate involvement of caspase 3 and 7 in wound healing and tissue regeneration.

The downstream factors that enable caspase 3 to influence the tissue regeneration process was also studied. Experiments showed calcium-independent phospholipase A₂ (iPLA₂) is involved in the tissue regeneration process. The activation of iPLA₂ was shown by others to lead to increased synthesis and release of arachidonic acid and lysophosphocholine in apoptotic cells. Since arachidonic acid is a precursor to prostaglandin E₂, which is a known stimulator of stem cell growth, tissue regeneration, and wound healing, the present inventors theorized that it was also involved in caspase influenced tissue regeneration process. To test this theory, experiments were performed to determine the rate of arachidonic acid release from wild type and casp3−/− mouse embryonic fibroblasts. Experimental results showed that ionizing radiation induced a significant amount of arachidonic acid release from wild type MEFs. However, this release was significantly reduced in casp3−/− MEFs. In addition, western blot analysis also confirmed a caspase 3 dependent cleavage of iPLA₂.

Because arachidonic acid is a precursor for prostaglandin E₂, PGE₂ concentration in the supernatants of wild type and casp3−/− MEFs was examined before and after irradiation. Results showed a strong correlation of supernatant PGE₂ concentration with cellular caspase 3 status. Supernatant from wild type MEF had a higher background concentration of PGE₂ and showed a strong increase after radiation. In contrast, both background and radiation induced levels of PGE₂ in the supernatant from casp3−/− MEF cells were significantly lower compared to those of wild type cells. In addition to the above data, a role of iPLA₂ in mediating radiation-induced PGE₂ concentration was clearly demonstrated in other ways. For example, (1) shRNA-mediated knockdown of iPLA₂ significantly reduced both background and induced levels of PGE₂ in wild type MEF cells; and (2) over-expression of a constitutively active (truncated) iPLA₂ gene, which expresses a truncated protein that is identical to a putative enzymatically active caspase 3/7 cleavage product of iPLA₂ protein significantly restored both background and induced levels of PGE₂ in casp3−/− MEF cells. These results show that caspase 3 regulates PGE₂ levels in dying cells through activation of iPLA₂. An additional experiment confirmed that PGE₂, instead of lysophophostidylcholine (LPC) (which are both products of activated iPLA₂), was responsible for growth stimulation mediated by iPLA₂.

Whether caspase 3-mediated iPLA₂ activation is important for stimulating stem/progenitor cell proliferation was also examined. This was done through the use of wild type MEF cells transduced with a shRNA-encoding gene against iPLA₂ and casp3−/−MEF cells transduced with a truncated but constitutively active version of iPLA₂ (that was identical to a previously identified caspase-generated fragment). These cells were evaluated for their ability to support epidermal keratinocyte progenitor cell growth. Results showed that reduction in iPLA₂ levels in wild-type MEFs significantly diminished their ability to support epidermal keratinocyte progenitor cell growth in vitro while transduction of the constitutively active iPLA₂ into casp3−/−MEFs significantly increased their capacity to support epidermal keratinocyte progenitor cell growth.

Results supporting a role of iPLA₂ in tissue regeneration were also obtained by use of the modified DIVAA tissue regeneration assay in nude mice. In this assay, host tissue in-growth into an artificially implanted silicone cylinders were quantified to determine the potency of cells/growth factors placed beforehand into the tube. Knocking down iPLA₂ expression significantly attenuated the ability of irradiated wild type MEF to induce host vascular growth into MEF cell-embedded silicone cylinders while exogenous expression of a constitutively active but truncated iPLA₂ significantly enhanced the ability of casp3−/− MEF cells to stimulate host tissue growth into the cylinders. These experiments provide a strong evidence for the importance of caspase 3-activated iPLA₂ in stem/progenitor cell proliferation and tissue regeneration. Similar results were also obtained in a similar assay using host mice that express green fluorescence protein.

It is surprising and unexpected that caspases (such as caspases 3 and 7), the “executioner” proteases that are usually considered instruments of cellular death functioning at the terminal stages of apoptosis, are involved in mobilizing tissue stem/progenitor cells and promoting tissue regeneration.

### Induced Pluripotent Stem Cells Production

Currently, the molecular mechanisms involved in the derivation of induced pluripotent stem cells (iPSCs) from differentiated cells are poorly understood. The present inventors have discovered that caspases (such as caspases 3 and 8), which are proteases associated with apoptotic cell death, play a role in induction of iPSCs from differentiated cells, e.g., human fibroblasts. Caspases 3 and 8 activation occurs soon after transduction of iPSC-inducing transcription factors. It was also discovered by the present inventors that oct-4, a iPSC transcription factor, is responsible for the activation. Inhibition experiments of caspase 3 or 8 in human fibroblast cells significantly or completely prevented the induction of iPSCs, respectively. Furthermore, activation of caspases 3 and 8 led to efficient caspase cleavage-mediated inactivation of retinoblastoma susceptibility (Rb) protein, which was discovered to be an important barrier for iPSC formation. Inhibition of Rb can significantly boost the efficiency of iPSC induction from human fibroblasts. Without being bound by any theory, it is believed that caspases play key roles in reducing or eliminating or overcoming epigenetic barriers to facilitate nuclear reprogramming in the iPSC derivation process.

Since the initial success in producing iPS cells from murine fibroblasts, a significant number of advances have been made. Validation of the process was provided by many independent studies. The authenticity of the embryonic stem cell like-properties of iPSC came from studies showing the ability of murine iPSCs to participate in formation of various tissues when injected into murine embryos at the blastocyst stage and to generate mice through tetraploid complementation. A further major milestone in the field was the successful derivation of iPSCs from human fibroblasts, which has great implications in human regenerative medicine. There are also numerous technological advances that were made, including replacement of one or more of the original transcriptional factors with chemical compounds, and the generation of iPSCs by use of recombinant proteins, which in principle causes no genetic modifications in host cell genome.

Despite the tremendous progress made in the iPSC field, the molecular mechanism(s) involved in the iPSC derivation process still remains unknown.

The present inventors theorized apoptotic caspases play a role in the iPSC induction process because they play a role in the reversal of the process, namely differentiation. To test theory, the present inventors developed two non-invasive caspase reporters to monitor activities of caspases 3 and 8. These two caspases were chosen because of their known involvement in differentiation. Caspase reporters developed by the present inventors consist of a firefly luciferase-GFP fusion protein (LucGFP) linked to a polyubiquitin domain. In between the two moieties, a caspase cleavage site (either -IETD- for caspase 8 or -DEVD- for caspase 3) was inserted. The assumption is that when caspases are inactive, the reporter proteins are recognized by proteasomes and degraded immediately because the polyubiquitin domain serves as tag for protein destruction by proteasomes. However, when either caspase 3 or 8 is active, the polyubiquitin domain is cleaved off the reporter protein, leading to enhanced GFP and luciferase signals because these proteins are now not subject to direct proteasome recognition and degradation.

The caspase reporters were transduced into human IMR90 fibroblasts via recombinant lentivirus vectors. After puromycin selection to ensure stable integration of the reporters, the iPSC reprogramming process was initiated by infecting caspase reporter-transduced IMR90 cells with recombinant lentivirus vectors carrying 4 transcription factors (Oct-4, Sox2, Nanog, and Lin28, or OSNL in abbreviation) that are known to induce iPSC formation. To avoid the use of myc, which is known to induce caspase activation and might confound the results, the OSNL protocol was chosen because it was shown not to be essential for iPSC induction.

Interestingly, increased caspases 3 and 8 reporter activities were observed from day 3 and continued to rise, and remained elevated during the rest of the observation period. To confirm the above imaging results, western blot analyses of caspases 3 and 8 were carried out in IMR90 cells transduced with OSNL. Results indicated that despite progressive increases in activated caspase 3 with time (as represented by cleaved caspase 3 levels), the levels of intact caspase 3 remained roughly the same in OSNL-transduced cells and in iPSCs, indicating a small and steady increase in overall caspase 3 protein levels. The level of activated caspase 3 remained robust in iPSCs. Interestingly, it was also quite significant in the H9 human embryonic stem cells (ESCs), similar to previous observations made in murine embryonic stem cells.

In comparison, there were significant increases in both total and activated caspase 8 levels in OSNL-transduced fibroblast cells. The levels of both remained significant during the entire course of observation. However, in contrast to caspase 3, there were minimal amounts of total and activated caspase 8 in iPSCs that emerged from OSNL-transduced IMR90 cells. H9 human embryonic stem cells displayed similar results.

Quantitative PCR analysis showed increased caspases 3 and 8 mRNA levels after OSNL transduction in IMR90 cells consistent with western blot analysis results. In IMR90-derived iPSCs and in human H9 embryonic stem cells, caspase 3 mRNA levels were about the same level as in IMR90 cells. However, caspase 8 mRNA levels in H9 hESCs or iPSCs dropped precipitously to about 1/10 of the level in IMR90 cells, again the results were consistent with western blot analysis.

Experiments with caspase reporters designed to determine which of the four iPSC-inducing factors (e.g. OSNL) is responsible for caspases 3 and 8 activation showed that Oct-4 was responsible for caspases 3 and 8 activation. Among the other three factors, Sox 2 and Lin28 had a little effect on caspases 3 and 8 induction, Nanog had a very small effect on caspases 3 and 8 activation (−1.5-2.0 fold). Therefore, it appeared Oct-4 is the factor that was primarily responsible for caspase 3 and 8 activation during OSNL-induced iPSC formation.

Western blot analysis confirmed that activities of both caspases 3 and 8 were induced by Oct-4 transduction. Oct-4 induced patterns of caspases 3 and 8 activation paralleled those induced by OSNL, confirming the primary role of Oct-4 in mediating OSNL-induced caspase activation.

Additional western blot analysis confirmed induction of caspases 3 and 8 activation in IMR90 cells transduced with the original “Yamanaka Factors” (i.e., OSKM, or Oct-4+Sox2+Klf4+Myc). A noteworthy difference is that OSKM appeared to induce caspases 3 and 8 activation at higher levels at day 10. Enhanced induction of caspases 3 and 8 activation is consistent with reports showing that myc, one of the four original Yamanaka factors, is able to induce caspase activation and cellular apoptosis.

To determine whether observed caspase activation in OSNL transduced cells causes apoptosis, TUNEL analysis was conducted to detect DNA fragmentation, which occurs at the end stage of apoptosis. Experiments showed that despite caspases 3 and 8 activation, there were small levels (1%-3%) of DNA fragmentation after OSNL transduction. In contrast, with the OSKM protocol, there was a significant amount of TUNEL staining after gene transduction. About 10%-20% of OSKM-transduced cells were TUNEL positive. This is consistent with the observation by others that myc could sensitize transduced cells to apoptosis in mammalian cells.

In order to determine whether the observed caspases 3 and 8 activation had any functional relevance in iPSC induction, down-regulation of caspase activities in OSNL-transduced cells was attempted. In the initial experiment, CrmA was we stably transduced through lentiviral vectors, a cow pox virus protein known to suppress caspases 1 and 8 activities, into IMR90 cells. Infecting these cells with OSNL-encoding lentiviral vector cocktail resulted in a significant (i.e., around 80%) suppression in the frequency of iPSC induction. This was a very surprising result because logically one would expect that inhibiting caspase activation would lead to increased iPSC generation because of reduced cell death.

Because CrmA inhibits both caspases 1 and 8, more specific approaches were used to determine which caspase was involved. First, a siRNA-based knockdown approach was used. In this approach, lentiviral vectors encoding either caspase 1- or caspase 8-targeted shRNA was used to infect IMR90 cells. After selecting for cells with stable shRNA knockdown (as verified by western blot analysis), they were infected with OSNL lentivector cocktail. Results showed that expression of shRNA targeted to caspase 8 suppressed about 55% of iPSC formation. In contrast, shRNA targeted to caspase 1 had no significant effect on iPSC induction frequency. These results show that the significant inhibitory effect of CrmA on iPSC formation is mediated through caspase 8. Similarly, the importance of caspase 3, which can be activated by caspase 8, was demonstrated by targeted knockdown of its expression through shRNA. A significant reduction (around 50%) in iPSC induction was observed in caspase 3 knockdown IMR90 cells.

The importance of caspase 8 was further demonstrated through transduction of the short version of the c-FLIP gene, which is a known cellular inhibitor of caspase 8. Stable transduction of c-FLIP(s) into IMR90 cells significantly attenuated frequency of iPSC induction (about 50% reduction), again confirming the importance of the caspase 8 in iPSC induction.

To provide further proof that the cleavage activities of caspases 3 and 8 are necessary for iPSC formation, lentiviral vectors encoding dominant-negative forms of caspases 3 and 8 were constructed. The dominant negative form of human caspase 8 gene (casp8-DN) encodes a virtually identical protein as the wild type except a single amino acid change (i.e. C360A), which disables its catalytic proteolytic function. This dominant negative form was cloned into a recombinant lentiviral vector. Similarly, the dominant negative form of caspase 3 (Casp3-DN), which contains a single mutation (C163A) that disables its proteolytic function, was also cloned into a lentiviral vector.

When IMR90 cells stably transduced with a lentiviral vector encoding casp8-DN were infected with the OSNL lentiviral cocktail, no iPSC induction process was observed. No iPSC clones were observed after 25 days of culture, compared with around 550 iPSC clones/5×10⁵ cells for wild type IMR90 cells. Besides further demonstrating the role of caspase 8 protein in iPSC induction, the significant inhibitory effect achieved through dominant negative caspase 8 expression also indicated that the cleavage activity of caspase 8 was important for iPSC induction.

Similar to caspase 8, it was found that stable transduction of a dominant negative caspase 3 (C163A) also significantly reduced the frequency of iPSC induction (about 80%). However, unlike the case of casp 8-DN a small amount of the induction process was observed, indicating that there is some redundancy for the function of caspase 3 but not for that of caspase 8 in the iPSC derivation process.

The importance of caspase 8 was also confirmed using the following experiments. Transduction of casp 8-DN together with OSKM suppressed iPSC induction almost completely (over 95% suppression) indicating the general importance of caspase 8 activation in different iPSC induction protocols.

These experiments indicate involvement of caspase 8 activation in iPSC induction by the transcription factor cocktails. The effect of exogenous caspase 8 expression on the iPSC induction process was determined. When IMR90 cells stably transduced with exogenous caspase 8 gene was used, the frequency of iPSC induction with the OSNL protocol showed a 50% increase when compared with control. Similarly, a 150% (or 2.5 fold) increase in iPSC induction was observed in IMR90-casp8 cells when the three-factor Yamanaka protocol (OSK) was used. These results again confirmed a key role for caspase 8 in the iPSC induction process.

Further experiments were conducted to verify the “stemness” of putative iPSCs induced through the OSNL+casp8 protocol. Results of the experiments showed that iPSC clones induced by use of the OSNL+casp8 protocol expressed various markers of ESCs. In addition, they can formed three germ layers when injected into SCID mice. Examination of the methylation status of the endogenous Oct-4 gene promoter of putative iPSCs further confirmed their close resemblance to ES cells, as did quantification of the mRNA levels of key ESC-specific genes. Further experiments also demonstrated that these cells could form embryoid bodies that express markers for a variety of differentiated cell types, consistent with properties of iPSCs.

Experiments were conducted to identify the mechanism through which caspase 8 facilitates iPSC induction. Because caspase 8 cleavage is necessary in facilitating the iPS process, it was reasoned that one or more of the cleavage substrates of caspases might be involved in the iPSC induction process. It was also reasoned that caspase 8 may function through deactivation of factors involved in maintaining the differentiated state of fibroblasts. Attention was focused on the retinoblastoma susceptibility (Rb) gene, which is a tumor suppressor gene that regulates cell cycle progression and has been reported to play a key role in promoting and maintaining cellular differentiation. It has been shown that activated caspase 8 can cause cleavage and deactivation of the Rb protein through the activation of caspase 3. In addition, inactivation of Rb through viral protein E1A or genetic deletion has been shown to facilitate cellular reprogramming to a stem cell-like state.

The role of Rb protein was investigated by probing its status during the iPSC induction process. Western blot analyses showed that Rb protein was intact before the transduction of OSNL. However, reduction in full-length proteins and increase in cleaved Rb proteins were obvious by day 3. By day 5, almost all full-length Rb proteins were absent. Interestingly, starting from day 10, an increase in both total and caspase cleaved Rb fragments were observed. Furthermore, in iPSCs as well as in H9 hESCs, Rb levels were similar to parental IMR90 fibroblast cells, indicating that Rb attenuation during iPSC was transient. The patterns of Rb cleavage during iPSC induction were almost parallel to the pattern of caspase 3 and caspase 8. In particular, activated caspase 3 levels correlated well with those of cleaved Rb, indicating a very close relationship between the two. However, despite continued presence of caspase 3 in iPSCs as well as in ESCs, there were only small levels of cleaved Rb in iPSC and ESCs. Rb proteins were present at full-length in iPSCs and ESCs at a relative very high levels. These results indicate that caspase-mediated Rb cleavage is necessary for iPSC induction but not for maintaining the stem cell state. Transduction of Oct-4 alone induced a similar Rb cleavage pattern, consistent with the role of Oct-4 in activating caspases 3 and 8.

Function of Rb in the iPSC induction process was also studied. This was done by cloning the human Rb gene into a lentiviral expression vector. This was then stably transuded into IMR90 cells. This experiment showed that over-expression of the Rb gene significantly reduced the frequency (about 50%) of OSNL-induced iPSC formation. These results show that caspase-mediated Rb inactivation is an important step for iPSC induction.

The importance of deactivating Rb was further demonstrated through shRNA-mediated Rb knockdown. When a shRNA targeted to Rb was stably transduced into IMR90 cells, the frequency of OSNL-induced iPSC formation was almost doubled, again consistent with Rb as an inhibitor of iPSC induction.

The inhibiting function of Rb was further confirmed through the use of human papilloma virus type 16 E7 (HPV16 E7) protein, which is known to inhibit Rb function. Co-transduction of E7 with OSNL increased the frequency of iPSC formation almost 5-fold. Furthermore, E7 was able to partially alleviate the complete blockade caused by Casp8-DN indicating that caspase 8-mediated reprogramming process at least partially goes through deactivating Rb. Rescue of iPSC formation in casp8-DN transduced cells correlated with almost complete degradation of Rb in IMR90 cells transduced with OSNL+casp8−DN+E7 suggesting the ability of E7 to destabilize Rb.

The causative relationship between caspases 3 and 8 activation and Rb cleavage was further confirmed in cells transduced with the dominant negative caspase 3 or caspase 8. Inhibition of caspase 8 activities partially blocked OSNL transduction-mediated Rb cleavage. On the other hand, a dominant negative caspase 3 almost completely blocked Rb cleavage after OSNL transduction. These data appears to indicate that caspase 3 activity was mostly responsible for Rb cleavage.

Without being bound by any theory, it is believed that Rb is a part of the multiple layers of “epigenetic walls” that protect the chromatin to maintain a differentiated state. In fact, it has been shown that Rb-deficient murine embryonic fibroblast cells spontaneously revert to an embryonic stem cell like state. Thus, it appears the roles of caspases as intracellular “wrecking crews” whose jobs in the iPSC induction process are to transiently dismantle that normally insurmountable “epigenetic walls” in differentiated cells. The importance of reducing or knocking down the epigenetic walls is underscored by the observation of the complete blockade of the iPSC induction process mediated by dominant-negative caspase 8 expression.

Additional objects, advantages, and novel features of this invention will become apparent to those skilled in the art upon examination of the following examples thereof, which are not intended to be limiting. In the Examples, procedures that are constructively reduced to practice are described in the present tense, and procedures that have been carried out in the laboratory are set forth in the past tense.

### EXAMPLES

### Example 1

These examples relate to treatment of tumor.

### Cell Culture

A variety of cancer and fibroblast cells were used in this study. Among these are the mouse breast cancer cell line 4T1, mouse fibroblast cell lines NIH3T3, human cancer cell lines MCF-7 (breast cancer line), MDA-MB231 (breast cancer line), HCT116 (colon cancer line), and human fibroblast cell strain IMR-90. All the above murine and human cancer lines are available from American Type Tissue Culture (ATCC, Manasas, Va., USA). In addition, wild type and caspase-deficient mouse embryonic fibroblast cells were obtained from Yale University (New Haven, Conn.). For maintenance of the cells, Dulbecco's Eagles's Medium (DMEM) (Invitrogen, Carlsbad, Calif.) supplemented with 10% fetal bovine serum was used to culture the cells.

Gene Transduction into the Cells

To transduce various exogenous genes into target cells, the lentivirus vector was used. Typically the pLEX system, a lentivector system purchased from Open Biosystems (Huntsville, Ala.), was used. Genes that were cloned into this vector include: the firefly luciferase gene (Fluc) obtained commercially from Promega (Madison, Wis.); a truncated, activated iPLA₂ gene obtained from RT-PCR. In addition, lentiviral vectors encoding shRNAs against caspase 3 or iPLA₂ were also used. All the lentiviral vectors were packaged into live lentiviral viruses in 293T cells according to manufacturer's instructions.

### Bioluminescence Imaging

For imaging luciferase, the IVIS200 instrument from Caliper Life Sciences (Hopkinton, Mass.) was used. For tissue cultured cells, luciferase signal was imaged by adding PBS or colorless OptiMEM medium (Invitrogen, Carlsbad, Calif.) with D-luciferin (Caliper Life Sciences, Hopkinton, Mass.) at a concentration of 0.15 mg/ml. The cells were imaged at a set time point (e.g., 10 minutes) after the administration of D-luciferin. This allowed the signals from different samples to be compared. After images were taken, the manufacturer supplied software was used to process the images for quantitative data.

Growth of Fluc-labeled tumor cells in vivo was followed through non-invasive bioluminescence imaging using the IVIS200 instrument (Caliper Life Sciences, Hopkinton, Mass.). Mice to be imaged were injected with 150 mg/kg of D-luciferin (obtained from Caliper Life Sciences) intraperitoneally in 200 μl of PBS and then anesthetized with continuous flow of isofluorane. Imaging of the mice was carried out 10 minutes later. The time between injection and imaging was kept constant among different batches of mice.

### Irradiation of Cells and Mice

X-ray irradiation of cells and mice were carried out in a RS-2000 Biological Irradiator. It was purchased from Rad Source Corporation (Atlanta, Ga.). The dose rate for the machine is about 1 Gy/minute.

### Establishing and Monitoring Tumor Cell Growth in Mice

Different approaches were used to establish tumor growth than traditionally adopted by other studies. In most of the studies, only a very small number of Fluc-labeled tumor cells (500-1000 cells each) were injected either alone or together with other unlabeled cells (either tumor cells or fibroblast cells) that were either irradiated or un-irradiated. Tumor growth in these cells was monitored through the quantification of bioluminescence signals emitted from labeled tumor cells by using the IVIS200 instrument following manufacturer's instructions.

### Measurement of Arachidonic Acid Release

To measure arachidonic release, cells in 6-well dishes were plated at a density of 1.0−2.0×10⁵ cells/well. About 1.0 μCi of [³H]-arachidonic acid (obtained commercially from GE Healthcare Life Sciences) was then added to the cells, which has about 1 mL of DMEM medium that was serum free and with 0.5 mg/mL of lipid-free bovine serum albumin (Sigma Chemical Co., St Louis, USA). After 16 hrs, the cells were washed 3× with fresh medium and incubated with 3 mL of DMEM medium supplemented with 5% serum. After another 5 hrs, when arachidonic acid in the supernatant reached a steady state level, the cells were exposed to 8-10 Gy of x-rays. Supernatants were then removed at 4, 24, and 48 hrs from the cells and counted with a scintillation counter for quantification of [³H]-arachidonic acid.

### ELISA Measurement of PGE₂

To evaluate the PGE₂ secretion from cells, about 2×10⁵ cells/well were plated in 6-well dishes. Cells were cultured in DMEM medium supplemented with 2% fetal bovine serum. They were then irradiated with x-rays (8-12 Gy). Supernatant from the cells were taken right before, and 24 hours and 48 hours after cellular irradiation. PGE₂ in the supernatants were then measured by using an ELISA kit purchased commercially from R&D Systems (Minneapolis, Minn., USA).

### Molecular Cloning

The pLEX lentiviral vectors system was used to deliver reporter and other genes into target cells. The system was obtained commercially from Open Biosystems (Huntsville, Ala., USA). The genes transduced through pLEX include: (1) firefly luciferase gene, which was transferred from the plasmid pGL4.31-luc2 from Promega (Madison, Wis.); (2) a truncated version of the mouse calcium-independent phospholipase A₂, which was amplified through RT-PCR from murine mRNA by use of the following primers:

```
Forward, 5′ G ACTAGT GCCACC ATGCAG C ACCAAGGACC TCTTCGACTG-3′
                              Spe I Kozak
Reverse, 5′ ATAAGAAT GC GGC CGC GTCCACGACCATCTTGCCCAG 3′
                                              Not I
```

The Pfx polymerase (Invitrogen, Carlsbad, Calif.) was used for the PCR amplification. The amplified fragment encoded aa453-679 (which is equivalent to aa514-733 of human iPLA2) of murine iPLA₂ (accession# NM-016915), which has been shown to be a constitutively active caspase cleavage product. The fragment was cloned into PCR-Blunt and excised with Spe I and Not I restriction enzymes and cloned into the Spe I and Not I sites of a modified pLEX plasmid. The modified pLEX plasmid has an influenza hemagglutinin (HA) tag inserted between Not I and Mlu I (two of the unique restriction sites in pLEX) so that genes inserted into the Not I site can be fused with the HA tag.

In addition, lentiviral vectors encoding shRNA-encoding minigenes targeted against the murine iPLA₂ gene were obtained from Open Biosystems (Huntsville, Ala.). These genes were carried in the pLKO.1 lentiviral vectors system. The one that showed a significant efficacy had the following targeting sequence:

```
CCGG CGTATGAAGGACGAGGTGTTT CTCGAG AAACACCTCGTCCTTCATACG TTTTTTG
          sense targeting seq. loop antisense targeting
                                                        seq. seq.
```

(catalog #Rmmu534-NM-016915 from Open Biosystems).

For knocking down the PGE2 receptor EP2, a commercially available vector from Open Biosystems (catalog #RMM3981-9594402) was used. The target sequence for this shRNA is:

```
CCGGGCTTTCACTATGACCTTCTTT CTCGAG AAAGAAGGTCATAGTGAAAGCTTTTT
        sense targeting seq. loop antisense targeting
                                                      seq. seq.
```

A caspase 3 targeted, shRNA minigene-encoding lentiviral vector was also constructed. The sequence of this shRNA is:

```
CGCGTCCCC ATGGGCATATGCATAATAA TTCAAGAGAT TATTATGCATATGCCCA
                        sense targeting loop seq. antisense
                                seq. targeting seq.
TTTTTTGGAAAT
```

A double stranded oligo containing the above shRNA sequence was cloned into the pLVTHM vectors, which is a lentiviral vector.

For dominant caspase 3, a key cystein was mutated in the catalytic domain of wild type murine caspase 3 (C163→A) through site directed mutagenesis. The mutant version was then cloned into the lentiviral vector pLEX.

In all cases, manufacturer's instructions or published protocols was followed to produce live, replication-deficient recombinant lentiviral vectors in 293T cells.

### Western Blot Analysis

Cellular lysates were obtained under most circumstances using the standard RIPA buffer. For running western blot analysis, 40-60 μg/per sample was used for gel electrophoresis. Samples were usually heated to 95° C. for 5 minutes before loading into electrophoresis gel. See Table I below for information of antibodies used in western blot analyses.

For cytochrome c western blot analysis, a digitonin-based buffer was used to lyse the cells. The buffer consisted of: 190 μg/mL of digitonin (obtained from Sigma Chemical, St Louis, Mo.), 75 mM NaCl, 1 mM NaH₂PO₄, 8 mM Na₂HPO₄, 250 mM sucrose. Treatment of cells with this buffer led to plasma membrane leakage of cytosolic material. After a brief spin, the supernatant were used to analyze for cytochrome c leakage into the cytosol through western blot analysis.

TABLE I
Antibodies used in this study for
western blot, IHC, and IF analysis.

| Target name | Antibody source | Clone info |
|---|---|---|
| Caspase 3 | Cell Signaling Technology | 8G10, rabbit monoclonal |
| (full length) | | |
| Caspase 3 | Cell Signaling Technology | 5A1E, rabbit monoclonal |
| (cleaved, | | |
| activated) | | |
| Caspase 8 | Cell Signaling Technology | 1C12, mouse monoclonal |
| Caspase 9 | Cell Signaling Technology | Rabbit polyclonal |
| Cytochrome c | Cell Signaling Technology | Rabbit polyclonal |
| iPLA₂ | Caymen chemical | Rabbit polyclonal |
| EP2 | Caymen chemical | Rabbit polyclonal |
| HA epitope | Roche Applied Science | 12CA5. Mouse monoclonal |
| SMA | Dako | 1A4, mouse monoclonal |
| GFP | Acam | Rabbit polyclonal (ab290) |

The pan caspase inhibitor z-VAD-fmk was obtained from EMD Bisciences (Gibbstown, N.J.). Cyclooxygenase inhibitor indomethacin and a long half-life version of PGE2, 16,16-dimethyl PGE2 (dmPGE2) were both obtained from Caymen Chemical (Ann Arbor, Mich.).

### Immunofluorescence and Immunohistochemistry Analysis

Antibodies were used for immunofluorescence analyses of the following proteins: casp 3, SMA, and GFP. Paraffin-embedded tumor tissues were used. For F4/80, sectioned frozen tumor tissues were used because of antibody functionality.

### Results

### Tumor Cell Repopulation Stimulated by Cell Death In Vitro

Experiments were conducted to examine if dying tumor cells could stimulate the growth of living tumor cells. In order to simulate in vivo scenarios where the vast majority of tumor cells are killed by radiation or chemotherapy, a small number (about 500) of firefly luciferase (Fluc)-labeled murine breast cancer 4T1 cells were seeded onto a bed of a much larger number (2.5×10⁵) of unlabeled “feeder” 4T1 tumor cells that were irradiated with x-rays at different doses. Growth of the small number of labeled living cells was then monitored through non-invasive bioluminescence imaging. The results indicated that 4T1Fluc cells grew significantly faster when seeded onto dying cells than when seeded alone. In addition, there was a dose-dependent response from the feeder cells. Non-irradiated feeder cells exhibited no noticeable supportive roles and those irradiated with higher radiation doses exhibited higher growth-enhancing ability.

In addition to radiation, other cytotoxic treatments, such as etoposide (VP-16) and hyperthermic heating treatments could also engender significant growth-enhancing functions to dying cells, indicating that pro-growth properties were common among dying cells. Additional supporting evidence came from combinations of other dying vs. living cell types, which also showed growth-stimulating properties.

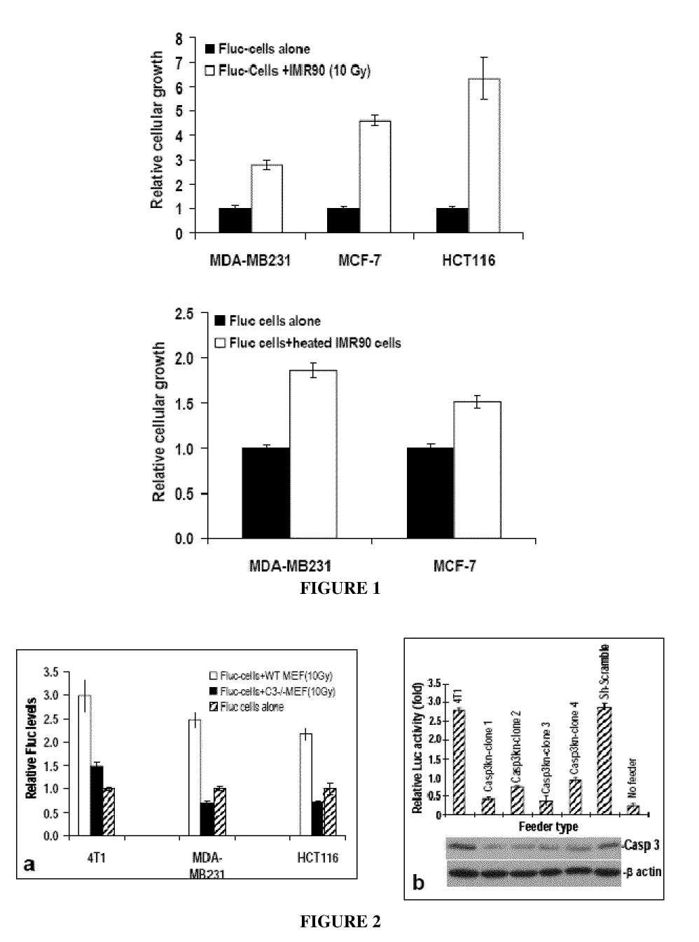

Because stromal cells in solid tumors play an important role in modulating tumor growth, dying fibroblast cells were evaluated to see whether they could promote tumor cell growth. Indeed, lethally irradiated mouse embryonic fibroblast cells stimulated the growth of different Fluc-labeled tumor cells significantly in vitro. Furthermore, similar results were obtained for human fibroblast cells that were either lethally irradiated or heated. See FIG. 1.

Thus, proliferation of a small number of tumor cells could be stimulated significantly by dying stromal fibroblast cells as well as tumor cells of mouse or human origins.

### Tumor Cell Repopulation Stimulated by Cell Death In Vivo

Whether cell death-stimulated tumor cell proliferation could be observed in vivo was examined. Briefly, a mix of untreated, Fluc-labeled and lethally irradiated, unlabeled tumor cells (at a ratio of 1:250, or 1000 live 4T1Fluc cells mixed with 2.5×10⁵ unlabeled, lethally irradiated 4T1 cells) were injected subcutaneously into the hind legs of nude mice. Subsequently the growth of the Fluc-labeled tumor cells was followed non-invasively over time through bioluminescence imaging. As controls, an equal number of Fluc-labeled 4T1 tumor cells mixed with live, unlabeled 4T1 tumor cells were injected into contra-lateral hind legs. The results showed that the presence of lethally irradiated tumor cells significantly increased the growth of Fluc-labeled tumor cells compared to Fluc-labeled tumor cells injected together with live tumor cells. The difference in the intensities of luciferase signals between the two groups grew exponentially larger and reached as great as 700 fold at the end of the experiment indicating a large difference in the growth rates of the two groups.

Similar in vivo tumor growth-promoting properties were also observed for mouse embryonic fibroblasts (MEF) that were irradiated. Fluc-labeled 4T1 co-injected with irradiated fibroblast cells grew to signal intensities 400 fold more than those from 4T1-Fluc cells injected alone in contra-lateral hind legs. These data indicate that dying stromal fibroblasts possess similar, potent growth-stimulating abilities as those of dying tumor cells.

### In Vitro Evidence for the Importance of Caspase 3 in Cell-Death-Stimulated Tumor Repopulation

Without being bound by any theory, it is believed that among the many cellular processes activated/deactivated in dying cells, factors/processes directly responsible for cell death are most likely to play key roles in regulating the growth-promoting properties of dying cells. Therefore, it is believed that caspases, the proteases that are involved in both the initiation and the execution of programmed cell death, are involved in regulating the growth-promoting properties of dying cells. To examine this theory, mouse embryonic fibroblast (MEF) cells with genetic deletion of their caspase 3 gene were obtained. The ability of these cells to support the growth of a small number of Fluc-labeled tumor cells was evaluated in similar assays as described above. Results (FIG. 2a) indicate that deficiencies in caspase 3 significantly compromised the ability of lethally irradiated MEF cells to stimulate the growth of Fluc-labeled murine (4T1) and human (MDA-MB231 and HCT116) tumor cells. The proliferation of Fluc-labeled tumor cells among the irradiated caspase 3 deficient (casp3−/−) cells was close to Fluc-labeled tumor cells seeded alone, indicating that caspase 3 was largely responsible for growth stimulation provided by dying cells.

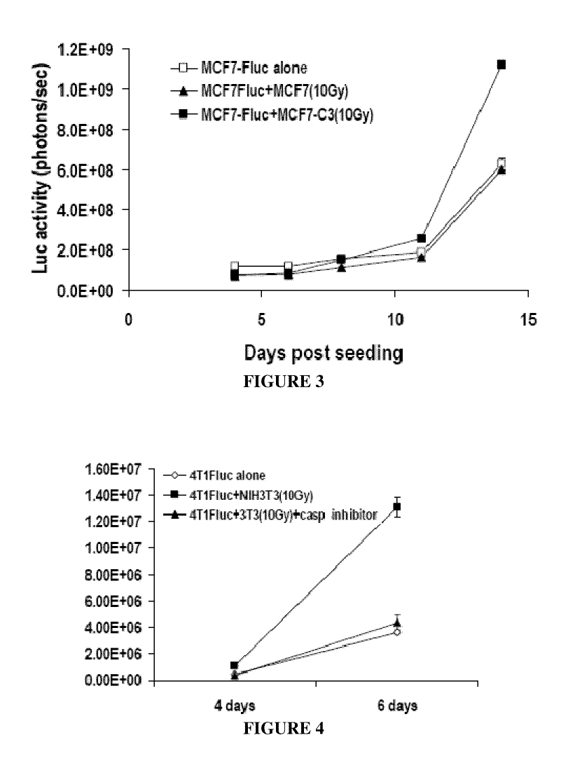

The importance of caspase 3 was also confirmed in lethally irradiated 4T1 cells. When caspase 3 gene expression was knocked down in 4T1 cells through lentivirus-mediated shRNA expression, the ability of lethally irradiated 4T1 cells to support Fluc-labeled 4T1 cell growth were significantly attenuated (FIG. 2b). The importance of casp3 was also confirmed in the human breast cancer cell line MCF7, which is deficient in casp3 expression. Exogenous expression of caspase 3 significantly increased the ability of lethally irradiated MCF-7 cells to promote co-seeded MCF-7Fluc cells (FIG. 3), again confirming the importance of caspase 3 in this process.

Clonogenic assays were conducted after wild type (WT) and casp3−/− MEF were exposed to different doses of x-rays. The results indicated that ionizing radiation kills both casp3−/− and wild type MEF cells to a similar extent. Therefore, the observed defect for casp3−/− MEF cells is not due to the lack of cell death in these cells after radiation. This conclusion is further supported by in vivo experiments where the growth of 4T1Fluc cells injected alone was compared with those injected together with irradiated casp3−/− MEF cells. The two populations of cell grew substantially at the same rate in vivo, indicating that there is no competition from the “undead” cells in among the irradiated casp3−/− MEF cells.

In order to rule out the possibility that caspase 3 mediates its growth-promoting function through a non-proteolytic mechanism, experiments were conducted using a pan-caspase inhibitor z-VAD-fmk to treat irradiated cells. The results showed that treatment of z-VAD-fmk eliminated the growth-promoting properties of irradiated cells (FIG. 4), indicating that caspase 3 activity is required for the observed growth-promoting properties of dying cells. A further, more definitive confirmation came from experiments where a dominant-negative version of caspase 3 was stably transduced into 4T1 cells. The dominant-negative caspase 3 (DN-casp3) differs with wild type caspase 3 in only one amino acid (C163A). However, it does not have the normal caspase cleavage capability despite its ability to bind substrates. The results showed that 4T1 cells transduced with DN-casp3 completely lost its ability to support the growth of 4T1Fluc cells. Therefore, growth-promoting properties of dying cells required the cleavage capabilities of caspase 3.

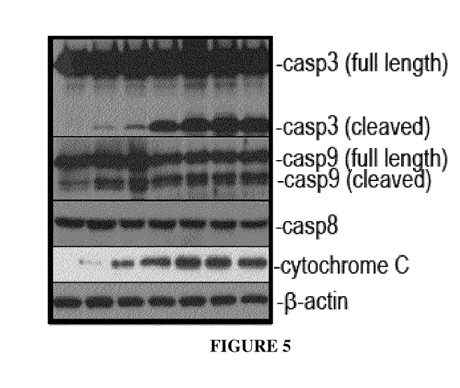

To further confirm that caspase 3 is activated in dying cells, a comprehensive immunoblot analysis of various proteins in the apoptotic pathway was performed. FIG. 5. The results showed that caspase 3 is activated in 4T1 cells in a dose-dependent manner. In addition, both cytochrome c and caspase 9 were activated in 4T1 and MEF cells while caspase 8 was not activated. These data indicated that in both irradiated 4T1 and MEF cells, the intrinsic apoptotic pathway was activated but not the extrinsic pathway.

### In Vivo Evidence for the Importance of Caspase 3 in Cell Death-Stimulated Tumor Repopulation

To examine the importance of caspase 3 in cell-death stimulated tumor repopulation in vivo, lethally irradiated MEF cells with different caspase 3 status were mixed together with a small number (500) of Fluc-labeled 4T1 cells and injected subcutaneously into nude mice. In contrast to potent stimulation of 4T1Fluc tumor cellular growth by lethally irradiated wild type MEF cells, significantly attenuated growth stimulation was observed for lethally irradiated caspase 3-deficient MEF cells. The difference between the two groups was as great as 1000 fold at later stages of observation. In fact, in separate experiments, 4T1-Fluc cells injected together with lethally irradiated caspase 3-deficient MEF cells grew at a similar rate as 4T1Fluc cells injected alone in the contra-lateral legs, indicating a lack of growth stimulation from casp3−/− cells.

The importance of caspase 3 in regulating growth-promoting properties of dying cells in vivo was also confirmed by co-injecting 4T1-Fluc cells with lethally irradiated 4T1 transduced with an shRNA minigene targeted against caspase 3. A significant reduction in the ability of lethally irradiated 4T1 cells to stimulate the growth of 4T1-Fluc cells were observed, consistent with the results obtained with casp3−/− MEF cells. Taken together, the above results indicate a role of caspase 3 in regulating the growth-promoting properties of dying cells.

Rapid Growth of Live Tumor Cells Injected into Irradiated Tumors.

Another series of experiments were conducted to simulate the behavior of a small number of surviving tumor cells in the radiotherapy-treated or non-treated tumor microenvironment. A small number of Fluc-labeled 4T1 tumor cells (about 1000) were injected into irradiated and non-irradiated tumors in the same group of mice and observed for growth. There was a clear difference in growth rates of the two groups. Cells injected into irradiated tumors grew at a significantly faster rate than cells injected into the non-treated tumors. These results were again consistent with the observation that dying cells in the tumor microenvironment stimulate the proliferation of surviving tumor cells.

In order to find direct evidence that caspase 3 is activated in irradiated tumors, 4T1 cells stably transduced with green fluorescence protein (GFP) was injected into irradiated and non-irradiated tumors and allowed to grow for 5-8 days. The mice were then sacrificed and tumors excised and examined for expression of various proteins. The results indicated a clear relationship between activated caspase 3 and the proliferation of injected, GFP-labeled tumor cells in irradiated tumor cells, as demonstrated by the complementary pattern of caspase 3 staining and injected tumor cell staining. In contrast, very little injected tumor cell growth and caspase activation were observed in non-irradiate tumors. In addition to caspase activation, irradiated tumors also showed increased, localized neovaculature. Thus indicating that caspase 3 is activated in vivo after exposure to radiation.

### Down-Stream Effector of Caspase 3 in Tumor Repopulation

Experiments were performed to identify the downstream factors of caspase 3 that were involved in generating growth-promoting factors from the dying cells. Among the large number of known caspase 3 cleavage targets, calcium independent phospholipase A₂ (iPLA₂) was examined because it had been shown that its phosopholipase activity was activated by caspase 3 cleavage. The activation was shown to increase production of arachidonic acid (AA), whose downstream eicosanoid derivatives (i.e., prostaglandin E₂), had been implicated in stimulating tumor growth and stem cell proliferation. To evaluate the potential involvement of caspase-activated iPLA₂-AA-PGE₂ process in cell death-induced tumor cell proliferation, lentiviral vectors that encoded a shRNA mini-gene against the iPLA₂ gene was transduced into 4T1 tumor cells and wild type MEF cells and examined to see whether these cells, when lethally irradiated, could still support Fluc-labeled tumor cell growth as much as their wild-type counterpart. In both MEF and 4T1 cells expression of an shRNA against iPLA₂ significantly reduced ability of these cells to stimulate Fluc-labeled 4T1 cellular proliferation. Western blot data also indicated that iPLA₂ was activated in a caspase 3-dependent manner in both 4T1 and MEF cells.

The involvement of caspase-mediated activation of iPLA₂ in tumor cell repopulation was further confirmed when a truncated version of the iPLA₂ gene (ΔiPLA₂), which encoded an iPLA₂ fragment (containing the catalytic domain) that was a predicted product of caspase 3 cleavage of iPLA₂, was transduced into caspase 3-deficient MEF cells. After ΔiPLA₂ transduction, lethally irradiated casp3−/− cells had significantly increased ability to stimulate the growth of Fluc-labeled 4T1-Fluc cells both in vitro and in vivo when compared with parental casp3−/− cells. Furthermore, measuring sizes of palpable tumors that emerge later in mice also confirmed the ability of a constitutively activated iPLA₂ in restoring the growth-stimulating ability of lethally irradiated caspase 3−/− MEF cells.

The importance of iPLA₂ was confirmed using in vivo experiments conducted with wild type MEF cells transduced with a shRNA gene against the iPLA₂ gene. Knocking down iPLA₂ gene had a significant attenuating effect on the ability of lethally irradiated MEF cells to stimulate the growth of 4T1-Fluc cells in vivo. In addition, measuring sizes of palpable tumors that emerge later in mice confirmed the effect of iPLA₂ knockdown to reduce the ability of lethally irradiated MEF to promote 4T1-Fluc growth.

Caspase 3 and iPLA₂ Regulate Cellular Arachidonic Acid and PGE₂ Release

Experiments were conducted to confirm the role of caspase 3 in regulating the activity of the iPLA₂ by examining its enzymatic activities. Because arachidonic acid is one of two main catalytic products of activated iPLA₂ (the other being lysophosphatidic choline, or LPC), radiation-induced release of arachidonic acid into the extracellular milieu was measured to examine potential relationship between caspase 3 and arachidonic acid. The results indicated that radiation stimulated the release of arachidonic acid into the supernatant significantly in wild type MEF cells. However, the release was significantly reduced in casp3−/− MEF cells, indicating a significant role for caspase 3. In addition, arachidonic acid (AA) release was also significantly reduced in 4T1 cells with effective knockdown of the caspase 3 gene expression. These results indicate a significant role of caspase 3 in regulating background as well as radiation-induced release of AA into the extracellular milieu.

Because arachidonic acid is the chemical precursor for prostaglandin E₂ (PGE₂), a key regulator of tumor growth that is generated from arachidonic acid via cyclooxygenase 1 and 2 (together with downstream action of PGE₂ synthase), it was believed that caspase 3-mediated activation of iPLA₂ would also lead to increased synthesis of PGE₂. Therefore, PGE₂ production induced by ionizing radiation was measured in the supernatant of cells. The results indicated that exposure to ionizing radiation significantly induced the production of PGE₂ in wild type MEF cells as well as in 4T1 tumor cells. However, in casp3−/− MEF cells and in 4T1 cells transduced with a shRNA against caspase 3, the production of PGE₂ in both background and irradiated cells was significantly reduced. In contrast, transducing a constitutively active, truncated iPLA₂ (which is equivalent to a caspase-cleaved version of iPLA₂), significantly restored PGE₂ production in caspase 3 deficient (casp3−/−) MEF cells. These results indicated a significant role of caspase 3-iPLA₂-AA axis in mediating radiation-induced PGE₂ production.

As PGE₂ had been implicated as a regulator of tumor growth, its effect on in vivo tumor growth was evaluated from a small number of Fluc-labeled 4T1 cells. The results indicated that treatment with PGE₂ can significantly stimulate tumor growth from a small number (about 1000) of subcutaneously injected Fluc-labeled 4T1 cells. The role of PGE₂ in tumor cell proliferation was also demonstrated by the fact that shRNA mediated down-regulation of EP2, a receptor for PGE₂, in 4T1Fluc cells significantly attenuated the proliferation of the latter when seeded together with lethally irradiated 4T1 cells.

### DISCUSSION

Surprisingly and unexpectedly, the present inventors have discovered that apoptotic tumor cells stimulate the repopulation of tumors from a small number of surviving cells. Even more surprising and unexpected is the finding that caspase 3, the master “executioner” during apoptotic cell death, serves as a direct link between cell death and tumor repopulation. Despite the paradoxical nature of these results at first glance, however, it is believed that the observed link between cellular death and proliferation is one of the key mechanisms of metazoan tissue homeostasis exploited by tumors to preserve themselves when damaged by cytoxic treatments.

Discovery by the present inventors, including a finding of an unexpected role for the apoptotic machinery in regulating tumor repopulation, shows that other factors that are involved in cell death, such as other caspases (other than caspase 3), and factors that regulates caspase activities (e.g., inhibitors of caspase activities such bcl-2, IAP proteins, and activators of caspase activities such as bax) may also be involved in the same process. Therefore, these additional molecular factors in the apoptosis can also be exploited to treat tumor. In view of the discovery by the present inventors as disclosed herein, drugs based on the apoptotic process can be used to treat various types of tumors.

For example, iPLA₂ is only one of the caspase targets that are activated by caspase-mediated cleavage. Many other caspase targets, such as various PKC variants, have been shown to be activated through caspase cleavage. It is generally believed that cleavage of these targets facilitates the apoptotic process. However, discovery by the present inventors indicates that the actual mechanism is more complicated than is currently understood. It is expected that some of the activated targets also play roles similar to iPLA₂.

One of the key biological processes in metazoan organisms is tissue regeneration through which wounded or damaged tissues are repaired. It is believed that caspases play a significant role in this process, similar to what the present inventors have demonstrated for tumor repopulation.

As disclosed herein, the present inventors have discovered that dying cells stimulate the proliferation of surviving tumor cells and subsequent repopulate tumors. This mechanism not only has profound implications in understanding cancer biology and treatment, it also provides new insights into how metazoan organisms repair tissue damage in general.

### Example 2

These examples relate to wound healing.

### Cell Lines and Culture Conditions

Epidermal keratinocyte progenitor (EKP) cells derived from C57BL/6 mice were purchased from Millipore (Billerica, Mass.). Neural stem cells (NSCs), derived from Balb/C mice, were purchased from American Type Culture Collection (Manassas, Va.). Mesenchymal stem cells (MSCs) were obtained from freshly isolated bone marrow following established protocols (Mesenchymal stem cells, Technical Manual, Stem Cell Technologies, and Vancouver, BC, Canada). In addition, wild type, caspase 3 knockout, caspase 7, and casp3−/−casp7−/− double knockout mouse embryonic fibroblast cells were obtained from Yale University (New Haven, Conn.). For culturing, EPK cells were maintained in Epidermal Keratinocyte medium from Millipore (catalog#CNT-02). NSCs were grown in neural stem cell medium (Millipore, catalog#SCM003). Wild type and caspase knockout MEF cells were cultured in DMEM medium with 5% FBS.

### Mouse Strains

Nude mice were obtained from National Cancer Institute (Bethesda, Md.). Wild type C57BL/6, caspase 3-knockout, and caspase 7 knockout mice, and EGFP-expressing transgenic mouse (all in C57/BL6 background) were obtained from the Jackson Laboratory (Bar Harbor, Me., US). The transgenic mice were deposited by Dr. Richard Flavell of Yale University. They were all in the C57BL/6 genetic background.

### Bioluminescence Imaging of Cultured Cells

Growth of Fluc-labeled stem/progenitor cells on lethally irradiated feeder cells were tracked though bioluminescence imaging. Imaging of the cells was carried out by use of an IVIS200 instrument from Caliper Life Sciences (Hopkinton, Mass.). Luciferin (obtained from Caliper Life Sciences) was added to the medium of cells to be imaged at a concentration of 150 μg/mL. After 5-10 minutes of incubation, petri dishes or multiwell plates were imaged in the instrument. Quantification of signal strength was carried out using manufacturer supplied software.

### Bioluminescence Imaging of Cells Implanted in Mice

Growth of Fluc-labeled stem/progenitor cells in vivo was also followed using non-invasive bioluminescence imaging using the IVIS200 instrument. Mice to be imaged were injected with 150 mg/kg of D-luciferin in 200 μL of PBS. Imaging of the mice was then carried out 10 minutes later. The time between injection and imaging was kept constant among different batches of mice.

### Skin Excision Wound Healing Assay

To characterize the rate of wound healing in different strains of mice, punch biopsy were created in the dorsal skin of mice by use of punch biopsy. The biopsy procedure was carried out using VisiPunch devices from Huot Instruments (Menomonee Falls, Wis., USA). Two 4 mm excision wounds were created in the dorsal skin of each mouse. Monitoring of the rate of healing was carried out by measuring the diameter of the wounds using a caliper every other day.

For histological analysis, skin biopsies containing the full-thickness wounds were taken from mice sacrificed at different time points after generating the excision wounds. About 60 minutes before the sacrifice, mice were injected with BrdU at 50 mg/Kg. They were then paraffin embedded.

For BrdU immunofluorescence staining, an Alexa Fluor 594 conjugated anti-BrdU mouse monoclonal antibody from Invitrogen (Carlsbad, Calif.) was used at a dilution of 1:20. To quantify the number of cells stained positive for BrdU staining, four randomly chosen 100× fields were counted.

To analyze skin epithelial cells and keratinocytes through immunofluorescence staining, antibodies against cytokeratin 14 and cytokeratin 6, respectively, were used. These were rabbit polyclonal antibodies produced in-house.

### Partial Hepatectomy-Liver Regeneration Assay

To quantify liver regeneration, a protocol of Mitchell et al. in Nat. Protoc., 2008, 3, 1167 was followed. Briefly, mice (8-10 weeks old) were anesthetized by use of isofluorane. After fixing the mouse, a 3-cm incision was created in the midline of abdominal skin and muscle to expose the xiphoid process. The median and left lateral lobes were tied around the top of the lobes using 4-0 silk surgical thread. The lobes were then removed surgically. This will lead to the removal of about ⅔ of the total liver weight. After suturing back the abdomen, the mice were placed on a warm pad to recover. At different times after hepatectomy, mice were sacrificed for evaluation. About 60 minutes before the sacrifice, BrdU (50 mg/kg) were injected i.p. into the mice to label proliferating cells.

Regeneration rate of the liver was quantified by use of two methods. In the first method, the weight of the two lobes of liver left intact in mice were determined and normalized to the weight of the same two lobes right after the initial surgery to derive a relative liver weight. In theory the range should be from 1.0 (right after surgery) to 3.0 (100% recovery). In the second method, immunofluorescence staining of BrdU positive cells was carried out in sections of paraffin embedded liver tissues. Liver regeneration was then quantified through enumeration BrdU positive cells in 4 randomly chosen 200× fields.

### Modified DIVAA Assay for Tissue Regeneration

To quantify the ability of different cells to induce tissue regeneration, a commercially available kit was adapted to measure tissue angiogenesis. The DIVAA in vivo angiogenesis assay) kit was obtained from Trevigen (Gaithersburg, Md.). The original purpose of this kit was to measure the ability of various growth factors to induced angiogenesis in vivo. The basic procedure involves mixing growth factors at different concentrations with basement membrane extract (BME) and loading the mixture (in a total volume of 20 μl) into a silicone cylinders called the “angioreactor”. The angioreactors were then implanted subcutaneously into nude mice by use of a simple surgical procedure. Up to 4 angioreactors can be implanted into a single mouse. The angioreactor has only one end sealed so that host tissue and vasculature can grow into it under proper conditions. In this experiment, instead of mixing growth factors with the basement membrane extract, various mouse embryonic fibroblast cells were mixed with BME. For each angioreactor, about 2×10⁵ cells were mixed. The total volume stays the same (20 μl). Two weeks after implantation, host mice were sacrificed and the angioreactors were taken out. The contents of the angioreactors were then transferred to microtubes and the endothelial cells content in them quantified by FITC-lectin staining following manufacturer's suggestion.

### Example 3

These examples relate to producing iPSCs.

### Construction of Caspase 3 and 8 Reporters

To construct the caspase 3 and 8 reporters, reporter plasmids were obtained from commercial sources (pEGFP-N1 from Clontech, Palo Alto, Calif.; and pGL2 from Promega, Madison, Wis.). Luciferase (without stop codon) was transferred into 5′ end of the EGFP gene within the pEGFP-N1 plasmid by PCR. During the PCR process, a caspase 3 (DEVD) or caspase 8 (IETD) recognition site was engineered into the 5′ end of the luciferase gene. In addition, a flexible linker (Gly₃Ser)₃ sequence was incorporated into the 3′ end of the luciferase gene. The stop codon for the latter was removed. Subsequently, a 9-unit polyubiquitin domain was obtained by use of RT-PCR from mRNA isolated from the human tumor cell line HCT116 and sequence-verified. It was then ligated into the 5′ end of luciferase gene. The fully assembled caspase 3 or caspase 8 reporter genes were then transferred from pEGFP-N1 into the lentiviral vector pLEX (Open Biosystems, Huntsville, Ala.).

Molecular Cloning of Caspases 3 and 8 and their Derivatives into pLEX Lentiviral Vector

Human caspase 3 and 8 genes and their dominant-negative versions were obtained from Addgene (deposited by Dr. Guy Salvesen from the Burnham Institute, La Jolla, Calif.). These genes were then transferred from their original plasmids into the pLEX lentiviral vector by PCR and subcloning (see below for a list of the primers used). In both (caspases 3 and 8), a hemaglutinin tag was inserted at the 3′ end to enable distinction between endogenous and exogenous proteins. For Rb (retinoblastoma), PCR was used to amplify and clone the full-length Rb gene (from IMR90-derived cDNA) with HA tag at the C-terminal end. For Rb fragments, the full-length Rb gene was used as a template in combination with PCR to obtain the coding sequence. In all cases, an HA tag is added at the C-terminal end. Primer information is listed below:

### Primer Sequences for PCR and Cloning

| Genes | Primer Sequences* |
|---|---|
| hCaspase 3 | Forward: 5′-gCTAGTGCCACCATGGAGAACACTGAAAACTC-3′ |
| | Reverse: 5′-gc ACGCGT TCGCGATCA AGC ATA ATC AGG AAC ATC |
| | ATA CGGATAAGCGGCCGCGTGATAAAAATAGAGTTCTTTTG-3′ |
| hCaspase 8 | Forward: 5′-cCTAGTGCCACCATGGACTTCAG CAGAAATC-3′ |
| | Reverse: 5′-ataagaatGCGGCCGCATCAGAAGGGAAGACAAG-3′ |
| Retinoblastoma | Forward: 5′-cACTAGTGCCACCATGCCGCCCAAAACCCCCCGA-3′ |
| (Rb) | Reverse: 5′-ataagaatGCGGCCGCTTTCTCTTCCTTGTTTGAGG-3′ |
| Rb fragments I | Forward: 5′-cACTAGTGCCACCATGC CGCCCAAAACCCCCCGA-3′ |
| (aa1-886) | Reverse: 5′-ataagaatGCGGCCGCatcTGCTTCATCTGATCCTTC-3′ |
| Rb fragment II | Forward: 5′-cACTAGTCCACCATGCCG GGAAGTAAACATCTCCCAG-3′ |
| (aa887-928) | Reverse: 5′-ataagaatGCGGCCGCTTTCTCTTCCTTGTTTGAGG-3′ |

*Sequences in italic indicate restriction enzyme cutting site (SpeI for sense and Not I for anti-sense). In small letters are protection nucleotides for restriction enzyme sites. In bold are sequences complementary to target genes. The underlined sequences are for adding HA tags.

### ShRNA Target Sequences

To knock down genes including caspases 1, 3 and 8, and Rb and Fas, lentiviral vectors (in either LKO or GIPZ backbone) were obtained from Open Biosystems (Huntsville, Ala.). Detailed targeting sequences are listed below:

shRNA Targeting Sequences

| Genes | shRNA sequences* |
|---|---|
| hCaspase 1 | 5′-TGCTGTTGACAGTGAGCGCGCAATCTTTAACATGTTGAATTAGTGAA |
| | GCCACAGATGTAATTCAACATGTTAAAGATTGCATGCCTACTGCCTCG |
| | GA-3′ |
| hCaspase 3 | 5′-TGCTGTTGACAGTGAGCGAGGAAACATTCAGAAACTTGAATAGTGA |
| | AGCCACAGATGTATTCAAGTTTCTGAATGTTTCCCTGCCTACTGCCTCG |
| | GA-3′ |
| hCaspase 8 | 5′-TGCTGTTGACAGTGAGCGAGGTCATCCTGGGAGAAGGAAATAGTGA |
| | AGCCACAGATGTATTTCCTTCTCCCAGGATGACCCTGCCTACTGCCTCG |
| | GA-3′ |
| hRb | 5′-TGCTGTTGACAGTGAGCGCGGATATAGGATACATCTTTAATAGTGA |
| | AGCCACAGATGTATTAAAGATGTATCCTATATCCTTGCCTACTGCCTCG |
| | GA-3′ |
| hFas | 5′-CCGGGCAAAGAGGAAGGATCCAGATCTCGAGATCTGGATCCTTCCT |
| (CD95) | CTTTGCTTTTTG-3′ |

*Underlined sequences indicate sense and anti-sense targeting sequences while bolded sequences indicate loop region in shRNA.

### Antibodies Used for Western Blot Analyses and Immunofluorescence Analysis

Antibodies against total and cleaved caspases 3, cleaved 8 are obtained from Cell Signaling Technology (Danvers, Mass.). The antibody against retinoblastoma (Rb, clone G3-245), full-length caspase 8, and Fas proteins were obtained from BD Biosciences (Billerica, Mass.). The antibody against HA (hemaglutinin) tag was obtained from Novus Biologicals (Littleton, Colo.). The antibody against β-actin (Pan Ab-5) was obtained from LabVision (Fremont, Calif.). Antibodies against the stem cell markers were obtained from Millipore (Billerica, Mass.)(TRA-1-81, SSEA-4, SSEA-3, and TRA-1-60) and AbCam (Cambridge, Mass.) (Oct-4, Nanog). Secondary antibodies (anti-mouse, rabbit, or goat) were obtained from Jackson ImmunoResearch (West Grove, Pa.). Secondary antibodies labeled with alexa fluor 488, or alexa fluor 594 were obtained from Invitrogen Corporation (Carlsbad, Calif.).

### Bisulfite Sequencing Analysis

To identify methylation status of CpG islands in the Oct-4 gene promoter, a published method of Yu et al, Science, 2007, 318, 1917-1920 was followed. Briefly, DNA from putative iPSCs was processed with EZ DNA Methylation-Gold Kit (Zymo Research Corp, Orange, Calif.) to convert unmethylated cytosines to uracil. Oct-4 promoter fragments were then amplified by PCR, cloned into TOPO PCR cloning vector (Invitrogen, Carlsbad, Calif.), and sequenced.

### Quantitative RT-PCR

To quantify gene activities at the transcriptional level, quantitative PCR analyses were conducted using a DNA Engine with Chromo 4 Q-PCR module (Biorad, Hercules, Calif.). Below is a list of some of the primers used for Q-RT-PCR. In addition, primers for Oct-4 and maker genes for cellular differentiation were the same as those published by Yu et al. in Science, 2007, 318, 1917-1920.

### Primers Used for Q-RT-PCR

| Genes | Primer sequences |
|---|---|
| hCaspase 3 | Forward: 5′-CAAACTTTTTCAGAGGGGATCG-3′ |
| | Reverse: 5′-TGA CAC GCC ATG TCA TCA TC-3′ |
| hCaspase 8 | Forward primer: 5′-CATCCAGTCACTTTGCCAGA-3′ |
| | Reverse primer: 5′-CATCCAGTCACTTTGCCAGA-3′ |

The foregoing discussion of the invention has been presented for purposes of illustration and description. The foregoing is not intended to limit the invention to the form or forms disclosed herein. Although the description of the invention has included description of one or more embodiments and certain variations and modifications, other variations and modifications are within the scope of the invention, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. A method for treating tumor in a subject comprising:
administering a therapeutically effective amount of a cytotoxic agent to the subject having a tumor to cause apoptosis of the tumor cells; and
administering a growth stimulating signal inhibitor to the subject to inhibit the growth stimulating signal released by the apoptotic tumor cells to reduce the amount of new tumor cell generation in the subject.

2. The method of claim 1, wherein the cytotoxic agent comprises a chemotherapeutic agent.

3. The method of claim 1, wherein the cytotoxic agent comprises electromagnetic radiation.

4. The method of claim 1, wherein the growth stimulating signal inhibitor is administered about 144 hours or less after the administration of the cytotoxic agent.

5. The method of claim 1, wherein the growth stimulating signal inhibitor is administered from about 1 hour to about 144 hours after the administration of the cytotoxic agent.

6. The method of claim 1, wherein the growth stimulating signal inhibitor comprises a caspase inhibitor.

7. The method of claim 6, wherein the growth stimulating signal inhibitor inhibits caspase-2, caspase-3, caspase-6, caspase-7, caspase-8, caspase-9, or a combination thereof.

8. The method of claim 7, wherein the caspase inhibitor comprises zVAD-fmk, z-DEVD-fmk, ac-YVAD-cmk DEVD-cho, and other known competitive or non-competitive caspase inhibitor, or a mixture thereof.

9. The method of claim 1, wherein the tumor is breast cancer, prostate cancer, colon cancer, melanoma, liver cancer, leukemia, lymphoma, and other solid tumors and blood-borne malignancies, or a combination thereof.

10. The method of claim 1, wherein the growth stimulating signal inhibitor is non-cytotoxic.

11. The method of claim 1, wherein the growth stimulating signal inhibitor is administered parenterally, orally, nasally, buccally, intravenously, intramuscularly, subcutaneously, intrathecally, transdermally, by osmotic pump, by inhalation, or a combination thereof.

12. The method of claim 1, wherein the growth stimulating signal inhibitor is administered at or near the tumor site.

13. A method for producing induced pluripotent stem cells (iPSC) from differentiated cells comprising subjecting differentiated cells to conditions sufficient to produce iPSC in the presence of:
(i) exogenous caspase-3, caspase-8, or a combination thereof;
(ii) a caspase activity promoter, wherein the caspase activity promoter increases the production or activity of caspase-3, caspase-8, or a combination thereof; or
(iii) a combination of (i) and (ii).

14. The method of claim 13, wherein differentiated cells are human fibroblast cells, keratinocytes, or a combination thereof.

15. A method for treating wound in a subject comprising administering a therapeutically effective amount of a wound healing composition to the subject to treat the wound, wherein the wound healing composition comprises:
(i) an exogenous caspase or a moiety having a similar protease activity;
(ii) a caspase activity promoter, wherein the caspase activity promoter increases the production or activity of one or more caspases; or
(iii) a combination of (i) and (ii)
wherein said method increases the rate of wound healing relative to the wound that is not treated with the wound healing composition.

16. The method of claim 15, wherein the caspase activity promoter increases the production or activity of caspase-3, caspase-7, caspase-8, or a combination thereof.


SEQUENCE LISTING (24 sequences)

SEQ ID NO: 1 - DNA, 40 bp, Artificial Sequence
  Forward Primer for murine mRNA
gactagtgccaccatgcagcaccaaggacctcttcgactg

SEQ ID NO: 2 - DNA, 37 bp, Artificial Sequence
  Reverse primer for murine mRNA
ataagaatgcggccgcgtccacgaccatcttgcccag

SEQ ID NO: 3 - DNA, 59 bp, Artificial Sequence
  shRNA-encoding minigenes targeted against murine iPLA2 gene
ccggcgtatgaaggacgaggtgtttctcgagaaacacctcgtccttcatacgttttttg

SEQ ID NO: 4 - DNA, 57 bp, Artificial Sequence
  Target sequence for knocking down PGE2 receptor EP2
ccgggctttcactatgaccttctttctcgagaaagaaggtcatagtgaaagcttttt

SEQ ID NO: 5 - DNA, 67 bp, Artificial Sequence
  caspase 3 targeted shRNA minigene-encoding lentiviral vector
cgcgtccccatgggcatatgcataataattcaagagattattatgcatatgcccattttttggaaat

SEQ ID NO: 6 - DNA, 32 bp, Artificial Sequence
  Forward primer sequence for hCaspase 3 gene
gctagtgccaccatggagaacactgaaaactc

SEQ ID NO: 7 - DNA, 76 bp, Artificial Sequence
  Reverse primer sequence for hCaspase 3 gene
gcacgcgttcgcgatcaagcataatcaggaacatcatacggataagcggccgcgtgataaaaatagagttcttttg

SEQ ID NO: 8 - DNA, 31 bp, Artificial Sequence
  Forward primer sequence for hCaspase 8 gene
cctagtgccaccatggacttcagcagaaatc

SEQ ID NO: 9 - DNA, 34 bp, Artificial Sequence
  Reverse primer sequence for hCaspase 8 gene
ataagaatgcggccgcatcagaagggaagacaag

SEQ ID NO: 10 - DNA, 34 bp, Artificial Sequence
  Forward primer sequence for Retinoblastoma gene
cactagtgccaccatgccgcccaaaaccccccga

SEQ ID NO: 11 - DNA, 36 bp, Artificial Sequence
  Reverse primer sequence for Retinoblastoma gene
ataagaatgcggccgctttctcttccttgtttgagg

SEQ ID NO: 12 - DNA, 34 bp, Artificial Sequence; identical to SEQ ID NO: 10
  Forward primer sequence for Rb gene fragment I

SEQ ID NO: 13 - DNA, 37 bp, Artificial Sequence
  Reverse primer sequence for Rb gene Fragment I
ataagaatgcggccgcatctgcttcatctgatccttc

SEQ ID NO: 14 - DNA, 37 bp, Artificial Sequence
  Forward primer sequence for Rb gene Fragment II
cactagtccaccatgccgggaagtaaacatctcccag

SEQ ID NO: 15 - DNA, 36 bp, Artificial Sequence; identical to SEQ ID NO: 11
  Reverse primer sequence for Rb gene Fragment II

SEQ ID NO: 16 - DNA, 97 bp, Artificial Sequence
  shRNA targeting sequence for hCaspase 1
tgctgttgacagtgagcgcgcaatctttaacatgttgaattagtgaagccacagatgtaattcaacatgttaaagattgcatgcctactgcctcgga

SEQ ID NO: 17 - DNA, 97 bp, Artificial Sequence
  shRNA targeting sequence for hCaspase 3
tgctgttgacagtgagcgaggaaacattcagaaacttgaatagtgaagccacagatgtattcaagtttctgaatgtttccctgcctactgcctcgga

SEQ ID NO: 18 - DNA, 97 bp, Artificial Sequence
  shRNA targeting sequence for hCaspase 8
tgctgttgacagtgagcgaggtcatcctgggagaaggaaatagtgaagccacagatgtatttccttctcccaggatgaccctgcctactgcctcgga

SEQ ID NO: 19 - DNA, 97 bp, Artificial Sequence
  shRNA targeting sequence for hRb
tgctgttgacagtgagcgcggatataggatacatctttaatagtgaagccacagatgtattaaagatgtatcctatatccttgcctactgcctcgga

SEQ ID NO: 20 - DNA, 58 bp, Artificial Sequence
  shRNA targeting sequence for hFas
ccgggcaaagaggaaggatccagatctcgagatctggatccttcctctttgctttttg

SEQ ID NO: 21 - DNA, 22 bp, Artificial Sequence
  Forward primer sequence for QRT-PCR of hCaspase 3
caaactttttcagaggggatcg

SEQ ID NO: 22 - DNA, 20 bp, Artificial Sequence
  Reverse primer sequence for QRT-PCR of hCaspase 3
tgacacgccatgtcatcatc

SEQ ID NO: 23 - DNA, 20 bp, Artificial Sequence
  Forward primer sequence for QRT-PCR of hCaspase 8
catccagtcactttgccaga

SEQ ID NO: 24 - DNA, 20 bp, Artificial Sequence
  Reverse primer sequence for QRT-PCR of hCaspase 8
catccagtcactttgccaga